(19) United States
(12) Patent Application Publication
(10) Pub. No.: US 2024/0024369 A1
(43) Pub. Date: Jan. 25, 2024

(54) ISOLATED PRIMARY DERMAL FIBROBLASTS FOR TREATING SKIN CONDITIONS

(71) Applicant: KING'S COLLEGE LONDON, London (GB)

(72) Inventors: Fiona Watt, London (GB); Christina PHILIPPEOS, London (GB); Magnus LYNCH, London (GB)

(21) Appl. No.: 18/283,660

(22) PCT Filed: Mar. 24, 2022
(86) PCT No.: PCT/EP2022/057831

§ 371 Date: Sep. 22, 2023

(30) Foreign Application Priority Data
Mar. 24, 2021 (GB) 2104130.6

(51) Int. Cl. A61K 35/33; C12N 5/077; A61K 9/00; A61P 17/02

(52) U.S. Cl. CPC A61K 35/33; C12N 5/0656; A61K 9/0014; A61P 17/02; C12N 2501/734; C12N 2501/73

(57) ABSTRACT

Provided herein is method of treating a skin condition in a subject, comprising isolating cells comprising primary dermal fibroblasts from a skin sample obtained from the subject, and administering the freshly-isolated cells to skin of the subject. Also provided are freshly isolated cell preparations comprising primary dermal fibroblasts and uses thereof.

### FIELD OF THE INVENTION

The present invention relates to the field of dermatology, and in particular to the role of fibroblasts in skin conditions such as ageing, wrinkling, scarring and dermal atrophy. The invention relates more specifically to methods for isolating cells comprising dermal fibroblasts from skin, and to the use of such isolated cells in medical and cosmetic methods.

### BACKGROUND OF THE INVENTION

The human skin is composed of a thin outer lining, the epidermis, that functions as a barrier and a tough thicker fibrous layer, the dermis, that gives the skin its volume and structural integrity. A wide range of disease processes can result in inflammation and fibrosis of the dermis with resultant scarring. One of the most challenging forms of scar to treat is the atrophic scar, where there is a reduction in the volume of the dermis with a resulting depression in the surface of the skin. This is particularly common with acne, however can also be caused by other inflammatory skin diseases, by surgery, trauma or self-harm. At the present time there are few effective treatments for atrophic scars. Smaller atrophic scars can be excised (surgically removed and stitched), however larger scars are more challenging to treat. Whilst dermal fillers can lead to a temporary improvement in appearance, they need to be repeated on a regular basis.

A characteristic feature of chronological aging is atrophy (thinning) of the dermis and this is associated with reductions in fibroblast cell number, alterations in the function of fibroblasts in maintaining the extracellular matrix and the formation of rhytides (wrinkles). There is a very large market for the treatment of these conditions. Due to the limitations of current therapeutic options such as dermal fillers for atrophic scars, age-related dermal atrophy and rhytides there have been efforts to develop fibroblast cell therapy products. A potential advantage of these products would be a long-lasting improvement in dermal atrophy in association with improvements in biomechanical properties of the skin such as elasticity without the need for repeat injections associated with dermal fillers. There is also the potential for harvesting fibroblasts from young individuals—either at the time of an unrelated cosmetic surgery procedure or during a skin biopsy and banking these cells cryogenically for re-introduction into the dermis at a later point in that individual's lifespan.

At the present time, the only FDA approved skin fibroblast cell therapy is Laviv (Fibrocell Science Inc). Following randomized controlled trials, this was approved for the cosmetic treatment of facial rhytides (wrinkles) in 2011 (Smith, et al., Dermatol. Surg. 2012 July; 38(7 Pt 2):1234-43.) with a subsequent trial in 2013 (Munavalli et al., Dermatol. Surg. 2013 August; 39(8):1226-36.) which showed efficacy in acne scarring. This treatment involves harvesting of a small skin biopsy from behind the ear from which fibroblasts are cultured. After 3 months these cultured fibroblasts are re-introduced in a series of injections every 3-6 weeks. Laviv has not proven successful commercially and has consequently been withdrawn from the market. This likely represents a combination of (i) the existence of safe and temporarily effective alternatives i.e. dermal fillers and (ii) the extended timeframe—3 months—between taking the initial biopsy and injecting the cells which would be logistically challenging for doctors and unappealing to many patients (iii) the high cost of culturing cells in GMP conditions for an extended period of time.

Accordingly there is a need for new methods for preventing or treating skin conditions using dermal fibroblasts. In particular, there is a need for more rapid and cost-effective methods for treating such conditions, that can be performed e.g. in a minor surgical facility in a single day.

### SUMMARY OF THE INVENTION

Accordingly, in one aspect the present invention provides a method of preventing or treating a skin condition in a subject, comprising isolating cells comprising primary dermal fibroblasts from a skin sample obtained from the subject, and administering the freshly-isolated cells to skin of the subject.

In one embodiment, the cells are administered to the subject without an ex vivo expansion step.

In one embodiment, the cells are administered to skin of the subject within 48 hours, 24 hours or 12 hours of obtaining the skin sample from the subject and/or isolating the cells from the skin sample.

In one embodiment, fewer than 500,000, 250,000 or 150,000 cells are administered per cm² of skin of the subject to be treated.

In one embodiment, the cells are administered to the subject in a medium further comprising calcium hydroxyapatite, synthetic poly-lactic acid microspheres, poly (methyl) methacrylate microspheres, polyalkylimide, fibrin, hyaluronic acid and/or collagen. In one embodiment, the cells are administered to the subject in a medium further comprising hyaluronic acid, and/or collagen and/or fibrin. In one embodiment, the cells are administered to the subject in an aqueous medium comprising or consisting essentially of fibrin, i.e. the cell preparation consists essentially of cells (preferably primary dermal fibroblasts) and fibrin in a liquid medium (e.g. aqueous solution).

In one embodiment, the cells are administered to the subject in a digestion medium used to isolate the cells, preferably without inactivation of enzymes that may be used in the isolation step (e.g. as described below).

In one embodiment, the method is a cosmetic method, and/or the skin condition is skin ageing, wrinkling, scarring or atrophy.

In one embodiment, the skin sample is obtained from the posterior auricular area, upper arm, thigh, abdomen or back of the subject, and/or the cells are administered to skin on the face, neck, hands or other body sites of the subject.

In one embodiment, the skin sample has an area of about 0.03 to 3 cm², preferably about 0.1 to 2 cm², more preferably about 0.5 to 1.5 cm².

In one embodiment, the cells are isolated from the skin sample by a method comprising one or more of the following steps: (a) (e.g. mechanical or enzymatic) removal of the epidermis from the skin sample; (b) enzymatic digestion of dermal tissue; and/or (c) separation of cells by cell filtration and/or centrifugation.

In one embodiment, the skin sample is incubated with one or more enzymes selected from dispase, collagenase, DNAse and/or hyaluronidase. In one embodiment, the skin sample is incubated with two or more enzymes, preferably two or more proteolytic enzymes, preferably two or more enzymes selected from dispase, collagenase, papain, trypsin, thermolysin, pronase, pancreatin, elastase, DNAse and/or hyaluronidase.

In one embodiment, one of the two or more enzymes comprises or consists of a collagenase.

In one embodiment, the percentage of fibroblasts in the freshly isolated cells comprises more than 30%, 50%, 60%, 70%, 80%, 90% or 95% of the total number of cells.

In one embodiment, the freshly isolated cells administered to the subject consist or consist essentially of fibroblasts.

In one embodiment, the cell isolation step further comprises separating dermal fibroblasts or a subpopulation thereof from the skin sample based on (e.g. positive or negative) expression of one or more cell-surface markers selected from CD26, CD31, CD39, CD36, CD45, CD90, CD324 and EpCAM, preferably CD90.

In one embodiment, the cell isolation step further comprises separating dermal fibroblasts or a subpopulation thereof from the skin sample using one or more ligands specific to a cell-surface marker of dermal fibroblasts immobilised on a solid phase, preferably one or more antibodies bound to a magnetic bead or column.

In one embodiment, the freshly-isolated cells comprise or consist of dermal fibroblasts, preferably having a cell surface phenotype CD45− CD31− CD324− CD90+; papillary dermal fibroblasts, e.g. having a cell surface phenotype CD90+ CD39+ CD26−; and/or reticular dermal fibroblasts, e.g. having a cell surface phenotype CD90+ CD39− or CD90+ CD39+ CD26+.

In one embodiment, the freshly-isolated cells show increased expression of one or more of the following genes compared to cultured cells: ADAMTS9, TCF4, CD39, Col11A1, LEF1, Col6A5; and/or the freshly-isolated cells show reduced expression of one or more of the following genes compared to cultured cells: CD90, DCN, Col1a2, FN1, LUM, PDGFRA, CD26, ADAM12, ELN, WNT5a.

In one embodiment, at least 100,000, 250,000 or 500,000 primary dermal fibroblasts are isolated per cm² of the skin sample.

In one embodiment, the subject is human.

In a further aspect, the present invention provides a freshly-isolated cell preparation comprising primary dermal fibroblasts obtained from a subject, for use in autologous prevention or treatment of a skin condition in the subject.

In one embodiment, the preparation is used to treat the subject without an ex vivo expansion step.

In one embodiment, the preparation is used to treat skin of the subject within 48 hours, 24 hours or 12 hours of isolation of cells from the subject.

In one embodiment, the preparation comprises fewer than 1,000,000, 500,000, 250,000 or 150,000 cells and/or wherein fewer than 1,000,000, 500,000, 250,000 or 150,000 cells are administered to the subject.

In one embodiment, the cell preparation further comprises one or more dermal fillers, preferably wherein the one or more dermal fillers comprise collagen, hyaluronic acid, calcium hydroxyapatite, synthetic poly-lactic acid microspheres, poly (methyl) methacrylate microspheres, polyalkylimide, and/or fibrin. In one embodiment, the preparation further comprises hyaluronic acid and/or collagen and/or fibrin. In one embodiment, the one or more dermal fillers consist of, or consist essentially of hyaluronic acid, fibrin and/or collagen.

In one embodiment, the preparation is used to treat a skin disorder or to promote wound healing.

In one embodiment, the preparation is for use by injection to the skin of the subject.

In a further aspect, the present invention provides a method for isolating cells comprising primary dermal fibroblasts from a skin sample obtained from a subject, comprising (a) removing the epidermis from the skin sample; (b) digesting dermal tissue in the skin sample with one or more enzymes; and (c) separating cells from the digested sample by filtration and/or centrifugation; wherein at least 100,000, 250,000 or 500,000 primary dermal fibroblasts are isolated per cm² of the skin sample.

In one embodiment, in step (b) the skin sample is incubated with collagenase type I, DNAse I and/or hyaluronidase. In one embodiment, in step (b) the skin sample is incubated with two or more enzymes, preferably two or more enzymes selected from dispase, collagenase, papain, trypsin, thermolysin, pronase, pancreatin, elastase, DNAse and/or hyaluronidase. In one embodiment, at least one of the two or more enzymes comprises a collagenase.

In one embodiment, the method further comprises separating dermal fibroblasts or a subpopulation thereof from the isolated cells based on (e.g. positive or negative) expression of one or more cell-surface markers selected from CD26, CD31, CD39, CD36, CD45, CD90 and CD324, preferably CD90.

In a further aspect, the present invention provides an isolated cell preparation obtained or obtainable by the method defined above.

In a further aspect, the present invention provides a pharmaceutical unit dosage form comprising freshly-isolated primary dermal fibroblasts in a liquid medium comprising one or more excipients, buffers, or diluents, wherein the unit dosage form comprises fewer than 1,000,000, 500,000, 250,000 or 150,000 cells.

In one embodiment, the unit dosage form comprises a sterile solution or suspension suitable for intradermal injection.

### BRIEF DESCRIPTION OF THE DRAWINGS

FIG. 1: Comparison of freshly isolated fibroblasts and cultured fibroblasts to support epidermal growth on human decellularized dermis (DED). Representative H&E-stained sections of 1 cm² DEDs, injected with freshly isolated or cultured human dermal fibroblasts (scale bars: 500 μm). Different cell numbers (100 000 and 500 000 cells) were tested for the freshly isolated sample. Experiments were performed with 2 technical replicates with donor cells derived from female facial skin, aged 97 (freshly isolated) and female abdominal skin, age 43 (passage 3, cultured fibroblasts).

FIG. 2: Quantification of epidermal thickness showing 100 000 freshly isolated dermal cells best supported epidermal reconstitution. Data are means±SEM. Statistical analysis was performed with ordinary one-way ANOVA with a Tukey multiple comparison test of the mean (**) p-value<0.005; (****) p-value<0.00005.

FIG. 3: Differential gene expression between freshly isolated and cultured dermal fibroblasts, analysed by RT-qPCR. Experiments were performed with donor cells derived from female skin, either freshly isolated cells or cultured fibroblasts after 3 passages). Experiment was performed with 4 technical replicates with values normalised to housekeeping genes (RPL13A and TBP) and presented as log 2 Fold Change. Data are means±SEM. Statistical analysis was performed with unpaired, two tailed t-tests (**) p-value<0.005; (***) p-value<0.0005; (****) p-value<0.00005.

FIG. 4: Upregulated gene expression in freshly isolated dermal fibroblasts compared to cultured dermal fibroblasts, analysed by RT-qPCR. Experiments were performed with donor cells derived from female facial skin, aged 97 (freshly isolated) and female abdominal skin, age 43 (passage 3, cultured fibroblasts). Experiment was performed with 4 technical replicates with values normalised to housekeeping genes (RPL13A and TBP) and presented as log 2 Fold Change. Data are means±SEM. Statistical analysis was performed with unpaired, two tailed t-tests (**) p-value<0.005; (***) p-value<0.0005; (****) p-value<0.00005.

FIG. 5. Histology of frozen dermis tissue digested with a) Collagenase (ColB), b) Collagenase and DNase I (ColB+DNase I), c) Collagenase, clostrapain and neutral protease (NB4), d) Collagenase, clostrapain, neutral protease and DNase I (NB4+DNase I), compared to e) a negative control treated only with PBS. Purple staining indicates non-digested tissue, blue staining indicates digested tissue. Scale bars=5 mm. Pie charts indicate the percentage of total tissue that was digested (blue) or non-digested (purple).

FIG. 6. Comparison of the number and viability of fibroblasts extracted from 6 mm of fresh, macerated dermis when using collagenase and papain, compared to collagenase, papain and trypsin.

### DETAILED DESCRIPTION OF THE INVENTION

In one aspect, the present invention relates to a method of preventing or treating a skin condition in a subject using autologous primary dermal fibroblasts. It has surprisingly been found that primary dermal fibroblasts can be obtained from a skin sample and used directly (without a cell culture step) to treat a skin condition in the same subject. Thus an in vitro or ex vivo cell culture expansion step is not required. This means that donor cells obtained from e.g. a skin biopsy taken from a subject can be used almost immediately to treat an affected area of skin on a different part of the subject's body. The method is therefore rapid and avoids the long delays (several weeks or months) required to expand donor cell populations in culture in previous methods.

The rationale for this method is based on several surprising findings. Firstly, sufficient primary dermal fibroblasts can be isolated directly from a small tissue biopsy to treat a significant area of skin in the same subject. Indeed it has been found that a much lower number of freshly-isolated primary dermal fibroblasts are required to reconstitute damaged skin than previously contemplated. Moreover, it has been found that uncultured (i.e. freshly-isolated) dermal fibroblasts are more effective in reconstituting skin tissue than cultured dermal fibroblasts. Together, these results demonstrate for the first time that uncultured dermal fibroblasts can be used as an effective autologous cell therapy for a range of skin conditions.

Obtaining a Skin Sample from the Subject

In a first step, a skin sample may be obtained from the subject. The subject is preferably a human subject, and may be affected by a skin condition. The skin condition is typically a localized condition, i.e. some areas of the subject's skin may be affected by the condition (but may be visible and/or cosmetically sensitive) whereas other areas are unaffected by the condition (but may be non-cosmetically sensitive).

Typically a small skin biopsy is taken, preferably from a non-cosmetically sensitive site. The skin sample site preferably has minimal skin appendages such as hair and sweat glands. For instance in some embodiments, the skin sample is taken from the posterior auricular area, upper arm, thigh or back of the subject. The lower back is a particularly suitable site since it has thick dermis (containing more fibroblasts), it is not sun-exposed and is not cosmetically sensitive being typically covered by underwear/swimwear.

The skin sample may be excised using standard techniques. Typically the area of skin around the target site is sterilised and may be injected with a local anaesthetic. A sterile scalpel may be used or suitably-sized punch biopsies taken.

The size of the biopsy sample is not limiting, and may be selected according to the size and nature of the area to be treated and the number of fibroblasts available in the biopsy sample. For example, it is noted that in hair transplantation procedures, a strip of skin of up to 10 by 1 cm may be harvested from the posterior scalp and it would be straightforward to harvest a similar quantity of skin from, for example, the lower back. However for most applications only a small number of fibroblasts will be required and the size of the skin sample may be e.g. about mm in diameter, e.g. 3 to 8 mm or 4 to 7 mm in diameter. A skin sample of approximately mm diameter corresponds to an area of skin of about 1 cm². Thus the skin sample may comprise an area of about 0.03 to 3 cm², preferably about 0.1 to 2 cm², more preferably about to 1.5 cm².

The area of the skin sample may also be varied based on the thickness of the dermis, since a thicker dermis will provide a greater yield of fibroblasts. For example, if the skin sample is taken from e.g. the lower back, which has a relatively thick dermis, a smaller skin area may be required than if the sample is taken from e.g. the face, which has a thinner dermis. A skilled person can easily select a suitable area of the skin sample from a particular site based on e.g. the known dermal thickness of particular skin regions (see e.g. Rook's Textbook of Dermatology, Ninth Edition, Ed. C. E. M. Griffiths et al., John Wiley & Sons 2016).

Isolating Primary Dermal Fibroblasts from Skin

In a next step of the method, cells are isolated from the skin sample. By this it is meant that e.g. the skin tissue is dissociated into its constituent cell populations, for instance in order to obtain an isolated cell preparation or suspension.

Firstly, the epidermis is preferably removed from the skin sample. This may be done e.g. by trimming off 1-2 mm of the upper surface of the skin sample with a sterile scalpel. Alternatively, an enzyme such as dispase may be used to digest the epidermis. In one embodiment, the skin sample is incubated with dispase, e.g. for 30-120 minutes at 35-40° C., preferably about 1 hour at 37° C. The epidermis may then be peeled off from the dermis following this incubation.

Dissociated cells may be obtained from the skin sample by enzymatic digestion of the dermis. Thus the dermis may be incubated with one or more enzymes, preferably two or more enzymes, selected from e.g. collagenase, DNAse, trypsin, papain, thermolysin, pronase, pancreatin, dispase (neutral protease), elastase, and/or hyaluronidase. The dermis may also be incubated with a proprietary tissue dissociation mixture such as Liberase™ TL, DL or TM. In embodiments, the dermis is incubated with at least a collagenase, preferably collagenase with one or more further enzymes selected from DNAse, trypsin, papain, dispase, elastase and/or hyluronidase. More preferably, the dermis is incubated with at least collagenase and papain, preferably collagenase, papain and trypsin. Preferably, the dermis is incubated with an enzyme mixture comprising collagenase type I, DNAse I and/or hyaluronidase. The combination of enzymes may vary depending on the donor site of the dermis and the biopsy size. In some cases, the enzyme mixture may be pre-prepared and may be e.g. re-constituted from a lyophilized enzyme preparation. The skin sample may be incubated with the enzyme(s) for a suitable time period, e.g. for 30-240 minutes at 35-40° C., preferably about 2 hours at 37° C.

An enzyme inhibitor (e.g. a metal ion chelating agent such as EDTA) may then be added to the digested dermal cell preparation. Typically the dissociated cells are then separated from the preparation by e.g. cell filtration and/or centrifugation. For instance, the cells may be filtered through a 50-150 μm cell strainer (e.g. about 70 μm or about 100 μm) and/or centrifuged at e.g. 200-400 g for 3 to 10 minutes. The cell pellet may be washed and resuspended in a suitable medium, e.g. phosphate-buffered saline (PBS).

In some embodiments, suitable methods to obtain an isolated cell preparation from skin samples such as those described in Collins et al., Development 138, 5189-5199 (2011) or Walmsley et al., Journal of Visualized Experiments, January 2016, 107:e53430 may be used. In alternative embodiments, a commercial kit or reagents such as those provided by CellnTec (Bern, Switzerland) or Miltenyi Biotec (Whole Skin Dissociation Kit; Bergisch Gladbach, Germany) may be used.

However in preferred embodiments, a method for isolating cells from a skin sample as described herein is used, e.g. as defined in Example 1. It is an advantageous feature of the methods described herein that a high number of primary dermal fibroblasts can be isolated from the skin sample. Preferably at least 100,000, 250,000 or 500,000 primary dermal fibroblasts are isolated per cm² of the skin sample.

The maximum number of cells yielded from a skin biopsy is restricted by safety of harvesting the biopsy, healing at donor site, and acceptability to patients. According to the methods described herein, fewer cells are required to achieve advantageous outcomes. For the first time, this means therapeutically meaningful cell numbers are within the range possible of generation by a bedside method. The methods described here permit rapid preparation of both autologous unfractionated fibroblasts and fibroblast subpopulations without the requirement for cell culture.

There has been a tacit assumption in the past that cell culture will be required to generate sufficient numbers of fibroblasts for clinically meaningful dermal transplantation. Previous studies have used 20 million fibroblasts in suspension, for human trials (Moon, AMA Facial Plast. Surg. 2019 Jul. 1; 21(4):312-318.). Using the present methods, it is expected that cell yield will typically be e.g. 63,500-127,000 papillary fibroblasts; and 254,000-508,000 reticular fibroblast enriched fractions per cm² skin. This analysis is based on fibroblasts isolated from breast or abdominal skin, which has a relatively thin dermis. The yield of fibroblasts is expected to be substantially higher when a donor site with thicker dermis, e.g. the back, is used. Thus in preferred embodiments, the skin sample is taken from a skin region with a relatively thick dermis, e.g. from a skin region that yields at least 100,000, 200,000 or 500,000 fibroblasts per cm² (e.g. the back).

### Cell Preparation Comprising Primary Dermal Fibroblasts

Thus the present invention provides a method for the preparation of primary dermal cells for cell therapy applications. This differs substantially from previous methods in that the cells can be prepared rapidly (within hours) in an outpatient facility without the requirement for cell culture. Applications of the cell preparation include, but are not limited to, the treatment of atrophic scars such as acne scars, rhytides (wrinkles), dermal atrophy as a consequence of disease processes and aging and non-surgical cosmetic procedures such as non-surgical rhinoplasty and zygomatic and chin enhancement. In comparison to previous methods this does not require culture of the cells and can be performed in a minor surgical facility whilst the patient waits.

Thus in embodiments of the present method, cells (i.e. a cell population or preparation) comprising primary dermal fibroblasts may be isolated from the skin sample. Typically the cell preparation is derived from a human skin sample, i.e. the cell preparation comprises human skin cells. In preferred embodiments, the cell preparation comprises dissociated cells, i.e. cells that have been separated from their original tissue environment and dispersed in a liquid medium. In one embodiment, the cell preparation comprises a cell suspension, e.g. a suspension of cells dispersed in a suitable aqueous buffer (e.g. PBS).

Typically the cell preparation (e.g. cell suspension) comprises primary dermal fibroblasts as well as other (i.e. non-fibroblast) cell types derived from skin, e.g. endothelial cells and/or leukocytes (e.g. lymphocytes, macrophages and/or dendritic cells). In some embodiments, dermal fibroblasts (or subpopulations thereof) may be separated from other skin types such as vascular endothelial cells and immune cells. Alternatively, the cell preparation may be administered to skin of the subject without a further separation step, i.e. the administered cell preparation may comprise primary dermal fibroblasts and one or more further skin cell types (e.g. endothelial cells and/or leukocytes).

Thus in one embodiment, the cell preparation comprises, consists essentially of, or consists of (dissociated) primary dermal fibroblasts in a liquid medium (e.g. a suitable solution or buffer). In another embodiment, the cell preparation comprises, consists essentially of, or consists of (dissociated) primary dermal fibroblasts, or a subpopulation thereof as defined herein, in a liquid medium (e.g. a suitable solution or buffer).

In preferred embodiments, the cell preparation contains a high proportion of primary dermal fibroblasts. Avoiding the presence of alternative cell types (e.g keratinocytes and/or melanocytes) may provide an improved safety profile, e.g. reduce a risk of undesirable effects from the non-fibroblast cell types. Therefore, in embodiments, the percentage of primary dermal fibroblast cells in the cell preparation is more than 30%, 40%, 50%, 60%, 70%, 80%, %, 90%, 91%, 92%, 93%, 94%, 95%, 96%, 97%, 98%, 98.5%, 99%, or 99.5% of the total number of cells. In preferred embodiments the cells of the cell preparation consist essentially of primary dermal fibroblasts, i.e. substantially 100% of the total number of cells in the cell preparation are primary dermal fibroblasts. In embodiments, the percentage of non-fibroblast cell types in the cell preparation is less than 50%, 40%, 30%, 20%, 15%, 10%, 9%, 8%, 7%, 6%, 5%, 4%, 3%, 2% or 1% of the total number of cells. The presence of too high a proportion of keratinocytes in the cell preparation may increase a risk of the formation of cysts at the application site (Watt (1984) J Cell Biol. 98(1): 16-21). Therefore, in embodiments, the percentage of keratinocytes in the cell preparation is less than 50%, 40%, 30%, 20%, 15%, 10%, 9%, 8%, 7%, 6%, 5%, 4%, 3%, 2% or 1% of the total number of cells. In preferred embodiments, the cell preparation does not contain keratinocytes. The presence of melanocytes in the cell preparation may increase a risk of uneven pigmentation at the application site (Zokaei et al. 2019. Cultured epidermal melanocyte transplantation in vitiligo: a review article. Iran J Public Health, 48(3): 388-399). In embodiments, the percentage of melanocytes in the cell preparation is less than 25%, 20%, 15%, 10%, 9%, 8%, 7%, 6%, 5%, 4%, 3%, 2% or 1% of the total number of cells. In preferred embodiments, the cell preparation does not contain melanocytes.

### Separating Primary Dermal Fibroblasts

In some embodiments of the present invention, the cell preparation may be enriched for primary dermal fibroblasts or subpopulations thereof, e.g. the dissociated primary dermal cells may be separated into subpopulations based on expression of specific cell surface markers. For instance, the dermal cells may be separated using methods as described in WO 2019/122931. In such methods, primary dermal cells are sorted or fractionated into subpopulations that express a particular cell surface phenotype, e.g. expression of particular markers and/or absence of expression of other markers. For instance, human dermal fibroblast subpopulations may be separated by selecting cells that express CD90, CD39, CD36 or CD26 individually, or by selecting human dermal fibroblasts that lack expression of CD90, CD39, CD36 or CD26 individually. In some embodiments human fibroblast subpopulations may be separated based on a combination of expression and absence of expression of two or more of the above markers.

In one embodiment, the dermal fibroblasts are separated by flow cytometry. For instance flow cytometry may be used to separate the dermal fibroblasts from other skin cells and/or to separate the human dermal fibroblasts into subpopulations. Fluorescence cytometry or fluorescence-activated cell sorting (FACS) is a specialized type of flow cytometry which is particularly useful for identifying and isolating cells according to surface markers, and may be used in one embodiment of the invention.

In alternative embodiments, the primary dermal fibroblasts or subpopulations thereof may be separated by any other method suitable for separating cells based on expression of cell-surface markers. In preferred embodiments, the cells are separated using a solid phase, e.g. a solid phase bound to a specific ligand. Methods using solid phase-based separation may be preferred to FACS in that solid phase separation may be easily and rapidly performed in a “bedside” manner, e.g. in an outpatient clinic with minimum specialist equipment. In contrast, FACS-based methods may require more time, specialized equipment and expertise and thus may be more difficult to perform rapidly in a standard clinical setting.

For instance, suitable solid-phase separation methods may involve the use of magnetic beads, columns and/or microfluidics. In such methods, a ligand (e.g. an antibody) specific for the cell-surface marker (e.g. CD90 or a marker of a fibroblast sub-population) may be immobilised on a solid phase (e.g. magnetic bead), and the cell population comprising human dermal fibroblasts in suspension contacted thereto. Human dermal fibroblasts expressing the marker of interest then bind to and are retained on the solid phase. Other cell types or human dermal fibroblasts lacking the marker do not bind to the solid phase and may thus be separated from the desired subpopulation by washing out the supernatant. The desired human fibroblast subpopulation may then be eluted from the solid phase if required.

In some embodiments, a negative selection step may be performed. For instance, a ligand (e.g. an antibody) specific for a cell-surface marker on undesired (e.g. non-fibroblast) cells in the preparation may be used. The undesired cells may be e.g. endothelial cells, leukocytes, keratinocytes, melanocytes or other non-fibroblast dermal cell types, and thus the cell-surface marker may be e.g. CD45, CD31 or CD324. Alternatively the undesired cells may be a specific subpopulation of dermal fibroblasts, and thus the cell-surface marker may be e.g. CD26, CD36 or CD39.

In such negative selection methods, the undesired cells may be removed e.g. by immobilizing the ligand (e.g. an antibody) specific for the cell surface marker on a solid phase, and contacting the cell population with the solid phase. The undesired cells are then retained on the solid phase and the dermal fibroblasts (or subpopulation thereof) are obtained from the supernatant. In alternative embodiments, the undesired cells may be lysed when bound to the ligand. For instance, the ligand may be an antibody that is capable of initiating complement-dependent lysis of cells. Such lysis-based methods may provide the further advantage of separating the desired dermal fibroblasts from undesired cells in solution, e.g. without requiring centrifugation or solid phase separation steps.

Thus in a preferred embodiment of the present invention, the cell population comprising primary dermal fibroblasts may be bound with one or more antibodies (e.g. a fluorescently-labelled antibody suitable for use in FACS, or an antibody bound to a solid phase or an antibody mediating solution-phase lysis of undesirable cell types). The antibody may bind directly to a human dermal fibroblast cell surface marker (e.g. CD90, CD39, CD36 or CD26), or may be a secondary antibody that binds to a primary antibody specific for the cell surface marker (e.g. a primary mouse IgG anti-human CD39, CD36 or CD26 antibody may bind directly to the human dermal fibroblasts, and a secondary fluorescent rat anti-mouse IgG antibody may bind to the primary antibody). Accordingly human dermal fibroblasts may be separated into subpopulations expressing a combination of markers including, but not limited to, a combination of markers selected from CD90, CD39, CD36 and/or CD26, e.g. using flow cytometry (e.g. FACS) or more preferably using a solid phase. Multi-colour fluorescence methods (e.g. using antibodies to different cell surface proteins labelled with fluorescent labels of a different wavelength) may be used to select human dermal fibroblasts based on expression of a combination of markers in a single FACS step.

Further specific markers of dermal cell populations are described e.g. in Reynolds et al., Science 22 Jan. 2021; Vol. 371, Issue 6527, eaba6500. In further embodiments, the cell preparation may be separated based on further cell-surface markers described therein. In addition, further (e.g. intracellular) markers of dermal cell populations as described therein may be used to validate the method, e.g. to confirm the identity and/or purity of the separated cell populations using nucleic acid-based detection methods (e.g. RT-PCR).

### Primary Dermal Fibroblasts

Primary dermal fibroblasts may be distinguished from other dermal cell types based on the expression of characteristic cell surface markers. For instance, in one embodiment CD90 expression may be used as a marker of human dermal fibroblasts. In other embodiments, human dermal fibroblasts may be identified as lin− CD90+ cells or lin− CD90− cells. Lin− CD90− cells present as a population of cells with mixed characteristics of pre-adipocytes, fibroblasts, pericytes and monocyte/macrophages. The lin− cell surface phenotype refers to cells lacking expression of CD45, CD31 and CD324. CD45 expression may be indicative of immune cells, CD31 is typically expressed on endothelial cells and CD324 may be expressed on keratinocytes. Therefore human dermal fibroblasts may be identified in one embodiment as e.g. CD45− CD31− CD324− CD90+ cells. In some embodiments, EpCAM (epithelial cell adhesion molecule) may be used as an alternative negative marker, e.g. the dermal fibroblasts may be identified as CD45− CD31− EpCAM− CD90+ cells.

Thus in one embodiment of the invention, human dermal fibroblasts may first be separated from other human skin cell types by selecting CD90+(e.g. CD45− CD31− CD324− CD90+) cells. The total human dermal fibroblast population may then be separated into subpopulations based on e.g. CD39, CD36 and/or CD26 expression, as described in WO 2019/122931. Alternatively, human dermal fibroblast subpopulations may be separated from other human skin cell types in a single step, e.g. by selecting CD90+ CD39+ CD26− (CD45− CD31− CD324− CD90+ CD39+ CD26−) cells, CD90+ CD39− (CD45− CD31− CD324− CD90+ CD39−) cells, CD90+ CD39+ CD26− (CD45− CD31− CD324− CD90+ CD39+ CD26+) cells, CD90+ CD36+(CD45− CD31− CD324− CD90+ CD36+) cells and so on. These selections may be performed, for instance, in one or more selection steps, e.g. using particular combinations of antibodies.

### Markers

In embodiments of the present invention, expression of one or more cell surface markers may be determined. In general, cells are determined as being either positive (+) or negative (−) for expression of each cell surface marker. By positive (+) it is typically meant that the cell expresses at least a minimum (e.g. detectable) level of the cell surface marker. For example, a cell may be considered to be positive (+) for the cell surface marker if an antibody specific for the cell surface marker is bound to the cell, e.g. in sufficient amounts that are detectable by flow cytometry (FACS) or such that the cell binds to a solid phase on which a ligand (e.g. antibody) for the cell surface marker is immobilized. Similarly, a cell may be considered to be negative (−) for a cell surface marker if it expresses the marker below a minimum (e.g. detectable) level. Accordingly a cell surface phenotype of a cell population may be designated based on expression and/or absence of expression of particular markers, e.g. CD45− CD90+ CD39−.

### CD26

CD26 is a cell membrane glycoprotein and serine exopeptidase expressed on the surface of various cell types. CD26 is also known as dipeptidyl peptidase-4 and adenosine deaminase complexing protein 2. The amino acid sequence of human CD26 is disclosed in, for example, database accession nos. P27487 (UniProt) and NP 001926 (NCBI RefSeq). CD26 may be detected by flow cytometry and CD26+ and CD26− cells sorted e.g. as disclosed in Kelemen et al., Am J Clin Pathol. 2008 January; 129(1):146-56.

### CD36

CD36 is a membrane protein that is a member of the class B scavenger receptor family. CD36 is also known as platelet glycoprotein 4, fatty acid translocase, scavenger receptor class B member 3 (SCARB3), glycoprotein 88 (GP88), glycoprotein IIIb (GPIIIB), or glycoprotein IV (GPIV). The amino acid sequence of human CD36 is disclosed in, for example, database accession nos. P16671 (UniProt) and NP_000063, NP_001001547, NP_001001548, NP_001120915 and NP_001120916 (NCBI RefSeq). CD36 may be detected by flow cytometry and CD36+ and CD36− cells sorted e.g. as disclosed in Cserti-Gazdewich et al., Cytometry B Clin Cytom. 2009 March; 76(2):127-34.

### CD39

CD39 is a cell surface-located ectonucleotidase also known as ectonucleoside triphosphate diphosphohydrolase-1 (ENTPD1). The amino acid sequence of human CD39 is disclosed in, for example, database accession nos. P49961 (UniProt) and NP_001091645, NP_001157650, NP_001157651, NP_001157653 and NP_001157654 (NCBI RefSeq). CD39 may be detected by flow cytometry and CD39+ and CD39− cells sorted e.g. as disclosed in Mandapathil et al., Journal of Immunological Methods, Volume 346, Issues 1-2, 31 Jul. 2009, Pages 55-63.

### CD90

CD90 is an N-glycosylated, glycophosphatidylinositol (GPI) anchored conserved cell surface protein also known as Thy-1 that is a member of the immunoglobulin superfamily. The amino acid sequence of human CD90 is disclosed in, for example, database accession nos. P04216 (UniProt) and NP_001298089, NP_001298091 and NP_006279 (NCBI RefSeq). CD90 may be detected by flow cytometry and CD90+ and CD90− cells sorted e.g. as disclosed in Nakamura et al., British Journal of Dermatology 154(6):1062-1070 (2006).

### CD45

CD45 is also known as leukocyte common antigen (LCA) and protein tyrosine phosphatase, receptor type, C (PTPRC). CD45 is a membrane glycoprotein expressed on almost all hematopoietic cells except mature erythrocytes. The amino acid sequence of human CD45 is disclosed in, for example, database accession nos. P08575 (UniProt) and NP_001254727, NP_002829 and NP_00563578 (NCBI RefSeq). CD45 may be detected by flow cytometry and CD45+ and CD45− cells sorted e.g. as disclosed in Janossy et al., Clinical and Vaccine Immunology 9(5):1085-1094 (2002).

### CD31

CD31 is a cell surface protein also known as platelet endothelial cell adhesion molecule 1 (PECAM-1). The amino acid sequence of human CD31 is disclosed in, for example, database accession nos. P16284 (UniProt) and NP_000433 (NCBI RefSeq). CD31 may be detected by flow cytometry and CD31+ and CD31-cells sorted e.g. as disclosed in Khan et al., Cytometry 64B(1):1-8 (2005) and Mock et al., Mucosal Immunology (2014) 7, 1440-1451.

### CD324

CD324 a cell-cell adhesion glycoprotein also known as cadherin-1, E-cadherin, CDH1 or uvomorulin. The amino acid sequence of human CD324 is disclosed in, for example, database accession nos. P112830 (UniProt) and NP_001304113, NP_001304114 and NP_001304115 (NCBI RefSeq). CD324 may be detected by flow cytometry and CD324+ and CD324− cells sorted e.g. as disclosed in U.S. Pat. No. 9,534,058.

### EpCAM

Epithelial cell adhesion molecule (EpCAM) is a transmembrane glycoprotein mediating Ca2+-independent homotypic cell-cell adhesion. EpCAM is expressed exclusively in epithelia. The amino acid sequence of human EpCAM is disclosed in, for example, database accession nos. P16422 (UniProt) and NP_002345 (NCBI RefSeq).

Antibodies to the markers discussed herein are known and typically available from commercial sources, or may be generated by known techniques such as immunization of experimental animals with a suitable antigen. Suitable antibodies for binding to such markers include, for example anti-mouse CD26 PerCP-Cy5.5 (eBioscience 45-0261-80), anti-human CD26 PE-Cy5 (Biolegend 302708), anti-human CD31 PE (eBioscience 12-0319-41), anti-human CD31 APC-Cy7 (Biolegend 303119), anti-human CD36 FITC (eBioscience11-0369-41), anti-human CD36 PE (Biolegend 336206), anti-human CD39 (eBioscience 14-0399-82), anti-human CD39 PE (eBioscience 1112-0399-41), anti-human CD39 APC (Biolegend 328210), anti-human CD45 AF700 (eBioscience 111256-9459-41), anti-human CD45 APC-Cy7 (Biolegend 368516), anti-human CD90 PE (eBioscience 12-0909-41), anti-human CD90 APC (Biolegend 328114), and anti-human CD324 PerCP/Cy5.5 (Biolegend 324113.

### Human Dermal Fibroblast Subpopulations

In the methods described herein, human dermal fibroblasts may be sorted into subpopulations based on the expression of any combination of the markers described herein, including any of the additional markers mentioned above. An isolated subpopulation of human dermal fibroblasts may be characterized by expression of a particular cell surface phenotype, e.g. CD45−CD90+CD39+CD26−. By “isolated” it is typically meant that the population of cells is separated from its natural environment, e.g. the subpopulation of human dermal fibroblasts is separated from the total population of human dermal fibroblasts and/or other dermal cells in the source sample (e.g. a human skin sample).

In one embodiment, the isolated cell population is characterized by expression of a cell surface phenotype CD90+ CD39+ CD26−, preferably CD45− CD31− CD324− CD90+ CD39+ CD26−. In one embodiment, the isolated cell population comprises human papillary fibroblasts.

In another embodiment, the isolated cell population is characterized by expression of a cell surface phenotype CD90+ CD39−, preferably CD45− CD31− CD324− CD90+ CD39−. In one embodiment, the isolated cell population comprises human dermal reticular fibroblasts.

In another embodiment, the isolated cell population is characterized by expression of a cell surface phenotype CD90+ CD39+ CD26+, preferably CD45− CD31− CD324− CD90+ CD39+CD26+. In one embodiment, the isolated cell population comprises human dermal reticular fibroblasts.

In another embodiment, the isolated cell population is characterized by expression of a cell surface phenotype CD90+ CD36+, preferably CD45− CD31− CD324− CD90+ CD36+. In one embodiment, the isolated cell population comprises human dermal pre-adipocyte fibroblasts.

In another embodiment, the isolated cell population is characterized by being vimentin+ and lin− CD90− but expressing CD74 (macrophage inhibitory factor receptor), HLA-DR and/or CLDN5 and TEK (TIE2). Typically, this subpopulation comprises cells with the ability to contribute to pre-adipocyte fibroblasts and pericytes.

### Freshly-Isolated Cell Preparation Comprising Dermal Fibroblasts

In embodiments of the present invention, the isolated cell preparation (i.e. comprising primary dermal fibroblasts or a subpopulation thereof) is used to treat a subject directly, e.g. freshly-isolated cells are used. By this it is meant that there is no cell culture expansion step, i.e. the primary dermal fibroblasts are not proliferated ex vivo or in vitro. Preferably the isolated cell preparation is used fresh after isolation and/or is not maintained ex vivo or in vitro for a significant time period before being used. The cells that are administered to the subject are thus uncultured cells.

In some embodiments, the steps of obtaining a skin sample and isolating cells from the skin sample may be performed rapidly, e.g. in less than 24 hours, less than 12 hours, less than 6 hours, or less than 4 hours. The freshly-isolated cells (i.e. comprising primary dermal fibroblasts) may thus be used to treat the subject within e.g. 24 hours, 12 hours, 6 hours, 4 hours or 1 hour of their isolation from the skin sample, e.g. from the beginning or end of the isolation step. Preferably the freshly-isolated cells (i.e. comprising primary dermal fibroblasts) are used to treat the subject within 48 hours, 24 hours, 12 hours, 6 hours or 4 hours of obtaining the skin sample from the subject.

In one embodiment, the freshly-isolated dermal cells are used without separation into subpopulations of fibroblasts. For instance, the freshly-isolated cells (e.g. total dermal cells or dermal fibroblasts) may be prepared as described above, but without a separation step into subpopulations of fibroblasts as described in WO 2019/122931. Thus the administered cells may comprise, consist essentially of or consist of total (i.e. unfractionated) dermal cells or total (i.e. unfractionated) dermal fibroblasts, e.g. without an ex vivo expansion step. An advantage of such methods may be that an additional separation step into subpopulations of fibroblasts may be avoided, and thus the method may be even faster and/or less complex.

In alternative embodiments, the freshly-isolated cells may be separated into subpopulations of fibroblasts, e.g. using one or more positive or negative selection steps as described above. For the avoidance of doubt, the isolated fibroblast subpopulations are still considered to be freshly-isolated since the selection steps can be performed relatively quickly (e.g. such that the isolated cells are administered within 48 hours of obtaining the skin sample).

### Additional Markers of Freshly-Isolated Dermal Cells

It has additionally been identified that freshly-isolated dermal cells show improved reconstitution of skin compared to cultured cells. Freshly-isolated (uncultured) dermal fibroblasts show differential gene expression compared to cultured fibroblasts. Thus upregulation and/or downregulation of expression of specific genes in freshly-isolated dermal cells may underlie their improved properties compared to cultured cells.

In some embodiments, the freshly-isolated cells show increased expression of one or more of the following genes compared to cultured cells: ADAMTS9, TCF4, CD39, Col11A1, LEF1 and Col6A5. In other embodiments, the freshly-isolated cells show reduced expression of one or more of the following genes compared to cultured cells: CD90, DCN, Col1a2, FN1, LUM, PDGFRA, CD26, ADAM12, ELN and WNT5a.

DCN, LUM, CD90 and PDGFRA are pan-fibroblast markers. COL6A5 is the a5 chain of collagen VI, and in particular represents a robust marker for papillary dermal fibroblasts. CD39, TCF4 and LEF1 may be markers of papillary fibroblasts that are lost when cells are cultured in vitro. Col11A1, Eln, FN1 and Col1a2 are extracellular matrix proteins (ECM) highly expressed in reticular dermal fibroblasts. ADAMTS9 and ADAM12 are members of the metalloendopeptidase protein family that regulates ECM, such as fibronectin fibrillogenesis and turnover. WNT5A is a regulator of fibroblast proliferation that is upregulated in culture. CD26 is a marker for a fibroblast subpopulation, as discussed above.

Additional markers of dermal cell populations as defined in Reynolds et al., Science 22 Jan. 2021; Vol. 371, Issue 6527, eaba6500 may also be used. These additional markers may comprise intracellular as well as cell surface markers, and may be used to further characterise the cell populations. For instance, in some embodiments the obtained cell preparation may be analysed for expression of one or more of the additional markers, e.g. as a validation step to confirm the suitability of the cell preparation to be used in treating the subject. In some embodiments, expression of the additional markers in the cell preparation may be compared to expression in a cultured cell preparation, i.e. after an ex vivo or in vitro expansion step.

Analysis of the additional markers may be performed by any suitable method for detection of the RNA and/or corresponding polypeptide sequences in the subpopulation of cells, e.g. RT-PCR, RNA-Seq, gene expression arrays, Northern/Western blots, ELISA and immunocytochemistry. Examples of primer pairs suitable for RT-PCR detection of some of the additional markers are shown in Table 1 below, with SEQ ID Nos. shown in brackets:

TABLE 1

| | Forward Primer | Reverse Primer |
|---|---|---|
| | 5′-3′ | 5′-3′ |
| Gene | (SEQ ID No.) | (SEQ ID No.) |
| Lef1 | ATCACACCCG | GGGGTGATCT |
| | TCACACATCC | GTCCAACACC |
| | (1) | (2) |
| Wnt5a | CGCGAAGACA | CGTATGTGAA |
| | GGCATCAAAG | GGCCGTCTCG |
| | (3) | (4) |
| Col6A5 | TGCGCTGAAC | AGCGTGGAAA |
| | CTTCGACTG (5) | TTGTCTGTTC |
| | | TG (6) |
| ADAMTS9 | AGCCATTCGA | ACATTGGGAA |
| | GAGTGCAACA | GCAGACCGTT |
| | (7) | (8) |
| TCF4 | TCCTCCAAAC | GCCCAACATT |
| | CAGCAACCAG | CCTGCATAGC |
| | (9) | (10) |
| CD39 (ENTPD1) | GCTGATTCCT | ATGTAGCCCA |
| | GGGAGCACAT | AAGTCCAGCC |
| | (11) | (12) |
| Col11A1 | TGGTGATCAG | AGGAGAGTTG |
| | AATCAGAAGT | AGAATTGGGA |
| | TCG (13) | ATC (14) |
| CD90 (Thy-1) | TCCCGAACCA | GATGCCCTCA |
| | ACTTCACCAG | CACTTGACCA |
| | (15) | (16) |
| DCN | AGAAGTTCCT | CGAAGATGGC |
| | GATGACCGCG | ATTGACAGCG |
| | (17) | (18) |
| Colla2 | AGGACAAGAA | GGTGATGTTC |
| | ACACGTCTGG | TGAGAGGCAT |
| | (19) | AG (20) |
| FN1 | TTACGGTGGC | CAGTGTGGTC |
| | AACTCAAATG | TGTGCAGAAA |
| | (21) | G (22) |
| LUM | GTGGTACCAG | TTGGGTAGCT |
| | TGGCCAGTAC | TTCAGGGCAG |
| | (23) | (24) |
| PDGFRA | TTCTGAACTC | GCTGCATCGG |
| | ACGGTGGCTG | GTCCACATAA |
| | (25) | (26) |
| CD26 (DPP4) | AGTGGCACGG | AGAGCTTCTA |
| | CAACACATT | TCCCGATGA |
| | (27) | (28) |
| ADAM12 | GTAGGCGTGG | ATGCTCATGA |
| | AAGTGTGGAA | TTGGGGCCAT |
| | (29) | (30) |
| ELN | TCCTGGAATT | AACTACTCCC |
| | GGAGGCATCG | GGGCCAAAG |
| | (31) | (32) |

A skilled person can easily identify alternative primer sequences suitable for specific amplification of the desired markers, using known methods. The nucleotide and amino acid sequences of the markers described herein, including various polymorphic variants thereof, are available from publicly-accessible sequence databases, as shown for example in Table 2 below:

TABLE 2

| | | Database accession numbers (human mRNA |
|---|---|---|
| Gene | Full name | sequence) |
| Lef1 | Lymphoid enhancer binding | NM_016269.4, NM_001166119.1, NM_001130714.2, |
| | factor 1 | NM_001130713.2 |
| Wnt5a | wingless-type MMTV integration | NM_001256105.1 |
| | site family, member 5A | NM_003392.4 |
| Col6A5 | collagen type VI alpha 5 chain | NM_001278298.1 |
| | | NM_153264.6 |
| ADAMTS9 | ADAM Metallopeptidase With | NM_182920, NM_003199, NM_001318781, |
| | Thrombospondin Type 1 Motif 9 | NM_001243227, NM_001243228, |
| TCF4 | Transcription Factor 4 | NM_001083962, NM_001243226, |
| CD39 | Ectonucleoside triphosphate | NM_001776, NM_001098175, NM_001164178, |
| (ENTPD1) | diphosphohydrolase 1 | NM_001164179, NM_001164181, NM_001164182, |
| | | NM_001164183, NM_001312654, NM_001320916 |
| Col11A1 | Collagen Type XI Alpha 1 Chain | NM_080680, NM_080681, NM_080679 |
| CD90 (Thy-1) | Thy-1 Cell Surface Antigen | NM_006288, NM_001311160, NM_001311162, |
| | | NM_001372050 |
| DCN | Decorin | NM_001920, NM_133503, NM_133504, NM_133505, |
| | | NM_133506, NM_ 133507 |
| Colla2 | Collagen Type I Alpha 2 Chain | NM_000089 |
| FN1 | Fibronectin 1 | NM_212482, NM_002026, NM_212478, NM_212476, |
| | | NM_001365524, NM_001365520, NM_001306129, |
| | | NM_001306132, NM_001306130, NM_001306131 |
| LUM | Lumican | NM_002345.4 |
| PDGFRA | Platelet Derived Growth Factor | NM_006206, NM_001347827, NM_001347828, |
| | Receptor Alpha | NM_001347830, NM_001347829 |
| CD26 (DPP4) | Dipeptidyl Peptidase 4 | NM_001935, NM_001379604, NM_001379605, |
| | | NM_001379606 |
| ADAM12 | ADAM Metallopeptidase Domain | NM_003474, NM_021641, NM_001288973, |
| | 12 | NM_001288974, NM_001288975 |
| ELN | Elastin | NM_000501, NM_001081752, NM_001081753, |
| | | NM_001081754, NM_001081755, NM_001278912, |
| | | NM_001278913, NM_001278914, NM_001278915, |
| | | NM_001278916, NM_001278917, NM_001278918, |
| | | NM_001278939 |

Expression of the above markers in uncultured (freshly-isolated) cells may be compared to expression levels in similar cells (i.e. cells obtained by a similar method) after a particular time period in culture, e.g. after 1 week, 2 weeks, 1 month or 3 months in culture. Alternatively marker expression in uncultured cells may be compared to expression levels in cultured cells after a particular number of passages, e.g. after 1, 2, 3, 4, 5 or 6 passages.

### Pharmaceutical Preparations and Unit Dosage Forms

The freshly-isolated cell preparations (i.e. comprising primary dermal fibroblasts) may be combined with any suitable pharmacologically acceptable excipients, diluents or carriers for administration to a subject. Typically the cell populations are formulated in a liquid medium, e.g. a suitable sterile buffer solution such as buffered Dulbecco's Modified Eagles Medium (DMEM) or sterile PBS. Pharmaceutical formulations comprising the isolated human dermal fibroblast subpopulations may be formulated for administration by any suitable route, preferably by injection, e.g. by intradermal injection into the skin of a subject.

A skilled person may determine a suitable unit dosage of the cell populations depending on the nature of the condition to be treated, the subject and so on. It has surprisingly been found according to the present invention that unit dosages as low as 100,000 cells may be particularly suited to reconstituting skin. Therefore a unit dosage form of the cell preparation preferably comprises less than 1 million, less than 500,000, less than 250,000 or less than 150,000 cells. In some embodiments, a single dose may comprise 10,000 to 1 million cells, preferably 20,000 to 500,000 cells, more preferably about 50,000 to 200,000 cells. Typically such a dose is administered (e.g. by injection) in 0.1 to 10 ml of solution, preferably 0.1 to 1 ml, e.g. about 100 μl of a suitable sterile buffer solution.

In some embodiments, the cell preparation may further comprise one or more dermal fillers, e.g. hyaluronic acid, collagen, calcium hydroxyapatite, synthetic poly-lactic acid microspheres, poly (methyl) methacrylate microspheres, polyalkylimide, and/or fibrin. Preferably, the dermal filler comprises or consists of hyaluronic acid, collagen and/or fibrin. More preferably the dermal filler consists of fibrin. Fillers may be derived from human, animals or synthetically produced. Hyaluronic acid is in widespread use as a dermal filler and has proven safe for the delivery of cultured fibroblasts to patients. Collagen may be e.g. human or bovine collagen. Collagen may be e.g. autologous (i.e., a collagen extracted from the patient's own skin) or collagen obtained from donors (e.g. Dermalogen). In further embodiments, the cells may be combined with an acellular scaffold, whether biological (e.g. decellularised human dermis) or inert (e.g. fibrin gel, collagen gel or a hydrogel). In a further embodiment the cells may be injected in the medium in which they were dissociated.

### Administration to the Subject

The isolated cell preparations described herein may be used in various cosmetic and therapeutic methods. In preferred embodiments, the freshly-isolated cells are administered directly to a subject, i.e. without ex vivo expansion. The isolated cell preparation is administered to a subject from which the skin sample was obtained, i.e. in an autologous cell therapy. Typically the isolated cell preparation is administered, e.g. by intradermal injection or through the use of a cannula to an area of skin in the subject that is affected by a skin condition (i.e., the ‘application site’).

It is characteristic of the present invention that a relatively low number of isolated cells is used to treat the subject, and that the isolated cells are administered to the subject soon after their isolation from the skin sample. Thus in some embodiments, fewer than 1,000,000, 500,000, 250,000 or 150,000 cells are administered per cm² of skin of the subject to be treated. Preferably about 10,000 to 1,000,000 cells/cm², more preferably about 20,000 to 500,000 cells/cm², more preferably about 50,000 to 150,000 cells/cm² and most preferably about 100,000 cells/cm² are administered to the subject. In such embodiments, the area of skin refers to the region which is affected by the skin condition and/or which is desired to be treated, e.g. the area of an atrophic scar or a region affected by wrinkles. Thus for example, in the case of an atrophic scar of area 2 cm², around 200,000 cells may be administered (e.g. via one injection or two injections of 100,000 cells each).

In preferred embodiments, fewer than 1,000,000, 500,000, 250,000 or 150,000 cells are administered per injection, e.g. 10,000 to 1,000,000 cells/injection, 20,000 to 500,000 cells/injection, 50,000 to 150,000 cells/injection or about 100,000 cells/injection. Most preferably the total number of cells administered to the subject (e.g. in a single round of treatment) is less than 1,000,000, less than 500,000, less than 250,000 or less than 150,000.

As discussed above, previous studies have used e.g. 20 million fibroblasts in suspension, for human trials. According to the present methods, e.g. 500,000 or fewer fibroblasts may be used, for instance in a medium such as hyaluronic acid.

The isolated human dermal fibroblast subpopulations described herein may be administered to a subject using known methods, e.g. as performed in existing clinical trials for human dermal fibroblast therapies. For instance, the cell populations described herein may be administered to a subject using methods described in clinical trials NCT01743053, NCT01115634, NCT02493816 and NCT00642642 (available from clinicaltrials.gov). Methods for performing fibroblast-based cell therapies are also reviewed in, for example, Leavitt T, et al., Scarless wound healing: finding the right cells and signals, Cell Tissue Res. 2016 September; 365(3):483-93 and Weiss R A, Autologous cell therapy: will it replace dermal fillers? Facial Plast Surg Clin North Am. 2013 May; 21(2):299-304.

### Therapeutic and Cosmetic Treatment of Skin Conditions

The isolated cell preparations may be used to prevent or treat various conditions. In general, as used herein the term “skin conditions” refers both to skin disorders and pathologies, as well as non-pathological skin conditions.

In one embodiment, the isolated cell preparation is used to treat a skin disorder. For example, the isolated cell preparation may be used to promote wound healing, or to treat keloidal or non-keloidal scarring, scleroderma, graft versus host disease, skin ulcers or genetic disorders such as epidermolysis bullosa.

The isolated cell preparation may also be used in various cosmetic methods, i.e. as a cosmetic treatment for a non-pathological skin condition. In a preferred embodiment, the isolated cell preparation is used to treat dermal atrophy. Thus the cell preparation may be used e.g. for preventing or treating skin ageing, wrinkles or scarring (e.g. atrophic scarring) in a human subject or for non-surgical cosmetic procedures such as rhinoplasty, zygomatic enhancement or chin enhancement or jawline refinement. Preferably the isolated cell preparation is administered to a cosmetically-sensitive area of the subject, e.g. to the face, neck or hands.

In one embodiment, the isolated human dermal fibroblast populations may be used to reduce fibrosis in the skin of a subject. For instance, the cell populations may be used to modify facial contour deformities such as nasolabial folds, glabellar crease, deep wrinkles of the forehead, and acne scars. In one embodiment, a freshly-isolated dermal papillary fibroblast subpopulation is used in such methods, e.g. an isolated population characterized by a cell surface phenotype CD45− CD31− CD324− CD90+ CD39+ CD26−.

One of the most challenging forms of scar is the atrophic scar. At present there are few effective treatments for atrophic scars. Small atrophic scars can be removed by surgery (excision and suturing), however larger scars are more challenging to treat. Whilst dermal fillers can lead to an improvement in appearance, they need to be repeated on a regular basis. An advantage of the present method over dermal fillers is that administering autologous cells capable of self-sustenance and replication, and not subject to clearance by an activated immune system, means therapeutic outcomes are long-lasting.

Prior attempts to introduce autologous fibroblasts have required the harvesting of fibroblasts followed by an extended period of culture for 3 months (see below). This is inconvenient for patients and logistically challenging for clinicians, moreover it is very expensive due to the inherent costs of maintaining cells for such a long period of time in a GMP culture facility. Whilst these costs may be economically viable for gene therapy applications they are unlikely to be feasible for the treatment of scarring or skin aging. An advantage of the present method is that fibroblasts can be prepared without the need for culture whilst the patient waits, dramatically reducing the costs and complexity of the procedure.

The effects of the cell preparations on skin conditions may be determined in suitable in vitro and in vivo models. For instance migration assays, in vitro explant cultures of biopsy wounds, extracellular matrix secretion assays, and in vivo excision wound healing models may be performed using the cell preparations in order to determine their effect.

The efficacy of the isolated cell preparations in the above therapeutic and cosmetic applications may be confirmed in preclinical and clinical studies. For instance, suitable models of keloid scarring based on analysing the activity of keloid-derived fibroblasts, including a keloid implantation animal model, are described in J. Liu, et al., Human adipose tissue-derived stem cells inhibit the activity of keloid fibroblasts and fibrosis in a keloid model by paracrine signaling, Burns (2017) [Epublication, http://dx.doi.org/10.1016/j.burns.2017.08.017]. The effect of isolated cell preparations according to the present invention in such models may also be determined. The treatment of keloid scarring in general is described in Ogawa R. Keloid and Hypertrophic Scars Are the Result of Chronic Inflammation in the Reticular Dermis. Int J Mol Sci. 2017 Mar. 10; 18(3). Thus suitable studies to determine the effects of isolated cell preparations in the treatment of keloid scarring may include in vitro keloid explants with fibroblast injections, collagen gel contraction assays, and proliferation assays of keloid fibroblasts treated with medium from freshly-isolated cell preparations.

### Kits

Also provided herein are kits for performing the methods described herein. Such kits may comprise, for example, apparatus and reagents for isolating cells comprising dermal fibroblasts from a skin sample as described above. For instance, the kit may comprise one or more of the following: a lyophilized enzyme cocktail, a digestion chamber, a cell filter, magnetic beads, an affinity column, and/or one or more buffers (e.g. PBS). The kit may comprise e.g. digestion, elution and/or washing buffers. An enzyme inhibitor solution (e.g. comprising a metal ion chelating agent such as EDTA) may also be present.

In some embodiments, the kit may comprise a reagent specific for one or more markers. Suitable reagents include ligands (e.g. antibodies) that bind specifically to the marker(s), or reagents such as oligonucleotide primers that direct specific amplification of marker sequences, e.g. mRNA or cDNA encoding the markers. The kits may comprise further reagents suitable for performing the detection method, e.g. reagents for ELISA assays such as secondary antibodies, buffer solutions or fluorescent labels or reagents for performing RT-PCR such as reverse-transcriptase, Taq polymerase, deoxyribonucleotides (dNTPs) and a suitable buffer. In preferred embodiments, the kit comprises a reagent (e.g. an antibody) specific for one or more markers bound to a solid phase, e.g. magnetic beads or an affinity column.

In a preferred embodiment, the kit comprises a combination of reagents specific for dermal fibroblast markers, e.g. CD90, CD39, CD36 and/or CD26. For instance, the kit may comprise antibodies or primers specific for CD39, CD36 and CD26. The kit may optionally further comprise reagents (e.g. antibodies or primers) specific for CD90, CD45, CD31 and/or CD324.

In further embodiments, the kit may comprise one or more further components for use with the obtained isolated cell preparation. For instance the kit may comprise one or more pharmaceutically-acceptable excipients, diluents or carriers for preparing an injectable pharmaceutical composition, e.g. a sterile (buffered) solution for injection. In another embodiment, the kit may further comprise a vial and/or injection device, e.g. a syringe and/or needle suitable for intradermal injection.

### Specific Methods

In one embodiment, the method comprises or consists of a number of steps as detailed below:
1. A small skin biopsy e.g. a 6 mm punch biopsy is obtained from a non-cosmetically sensitive site, such as the posterior auricular area, the upper arm, thigh or back.
2. The biopsy is placed in a single-use digestion chamber containing a lyophilized cocktail of collagenase enzymes; medium is added to reconstitute the enzyme mixture; and incubated in a single use incubator at 37° C. for 3 hours. This chamber may contain immobilized antibodies and a filter to trap undigested skin fragments.
3. The digested tissue is passed through a cell filter into a second single use chamber either containing magnetic beads (dynabeads) in suspension or conjugated to a column. The beads are conjugated to an antibody (e.g. anti-CD90, which binds to fibroblasts).
4. A magnet is applied to the outside of the chamber/column binding the magnetic beads and captured cells.
5. The column is flushed with PBS to remove all digestion enzymes, undigested ECM and cells that are not captured by the magnetic beads.
6. Cells are eluted from magnetic beads with an elution buffer and captured in a third chamber.
7. In an optional step additional positive and/or negative selection is performed with magnetic beads (e.g. CD39, CD26) to isolate previously identified subpopulations of fibroblasts.
8. In another optional step, cells are incubated with a lentiviral or similar transfection vector to effect genetic modifications.
9. Fibroblasts are resuspended in hyaluronic acid to facilitate efficient engraftment within the dermis following injection.
10. The hyaluronic acid filler containing resuspended fibroblasts is injected through a large bore needle into the dermis and a dressing is applied.

The invention will now be described by way of example only, with reference to the following non-limiting embodiments.

### EXAMPLES

### Example 1

Isolation of Freshly-Isolated Cell Preparation Comprising Dermal Fibroblasts from Human Skin Sample

A non-sun exposed area of skin with minimal presence of skin appendages (hair, sweat glands) is sterilised with iodine/chlorhexidine and injected with local anaesthetic. Three 4 mm punch biopsies are retrieved.

The epidermis is removed either by trimming off 1-2 mm of skin with a sterile scalpel, or by peeling off the epidermis following a 1 hour incubation in Dispase at 37° C., in a shaking water bath.

The dermal pieces are cut into quarters and incubated in 1 ml digestion medium (2.75 mg/ml Collagenase type I, 0.05% DNAse I; 1 mg/ml Hyaluronidase IV-S (750-3000 units/mg solid) 100 U/ml penicillin; 100 mg/ml streptomycin in DMEM (high glucose), filter sterilised) for 2 hours at 37° C., in a shaking water bath. Enzyme-inhibitor solution (500 μM EDTA in phosphate-buffered saline (PBS)) is added to the resulting cell suspension, filtered through a 100 μm cell strainer, and centrifuged at 300×g, for 5 minutes at 4° C.

The supernatant is disgarded and the pellet is washed once with 1.5 ml PBS at at 300×g, for minutes at 4° C. The pellet is resuspended in 0.2 ml PBS/saline and counted. Cell concentration is diluted to 1×10⁵ cells per 100 μl (1×10⁶ cells/ml) by the addition of the cell resuspension delivery solution/saline. The solution is gently mixed by pipette/vortexed at a very low speed to ensure cells are uniformly mixed, and into a 1 ml syringe capped with a luer-screw. The process takes less than 5 hours in total from taking the skin biopsy to obtaining an isolated population of cells.

The yield of dermal cells obtained using the method above from a number of biopsy samples from different donors is shown in Table 3 below:

TABLE 3

| No. of 4 mm | No. of cells | Cells per | Cells for 3 | Donor | Donor |
|---|---|---|---|---|---|
| Biopsies | isolated | biopsy | biopsies | age | site |
| 36 | 14150000 | 393056 | 1179167 | 62 | Breast |
| 12 | 3000000 | 250000 | 750000 | 22 | Breast |
| 24 | 9450000 | 393750 | 1181250 | 36 | Breast |
| 6 | 1600000 | 266667 | 800000 | 38 | Breast |
| 24 | 7600000 | 316667 | 950000 | 48 | Abdomen |
| 24 | 11500000 | 479167 | 1437500 | 42 | Abdomen |
| Average | 7883333 | 349884 | 1049653 | | |
| Standard | 4862373 | 87758 | 263274 | | |
| deviation | | | | | |

### Example 2

### Preparation of Cultured Cell Preparation Comprising Dermal Fibroblasts Maintained Ex Vivo

For a comparative study, primary donor cells were first obtained by isolation from a subject according to the method set out above, and then maintained in culture ex vivo.

Human adult dermal fibroblasts were cultured in Dulbecco's modified eagle medium (DMEM)+10% (v/v) FBS, 2 mM L-glutamine, and 100 U/ml penicillin, 100 μg/ml streptomycin (Gibco) or Amniomax C100 medium with Amniomax C100 supplement (Gibco). Culture flasks were incubated at 37° C. in a humidified atmosphere with 5% CO₂ and passaged every 3-5 days, when 80% confluent. Cells were used between passages 1-6 for all studies. Stock cultures of primary normal human keratinocytes (NHKs, strain km) were obtained from surgically discarded foreskin and grown on 3T3-J2 feeder cells. NHKs were used for DED experiments between passages 2-5. 3T3-J2 fibroblasts were originally obtained from Dr James Rheinwald (Department of Dermatology, Harvard Skin Research Centre, USA), not authenticated. All cell stocks were routinely tested for mycoplasma contamination and were negative. NHKs were cultured in complete FAD medium, containing 1 part Ham's F12, 3 parts DMEM, 10⁻⁴ M adenine, 10% (v/v) FBS, 0.5 μg ml⁻¹ hydrocortisone, 5 μg ml⁻¹ insulin, 10⁻¹⁰ M cholera toxin and 10 ng ml⁻¹ EGF, on mitotically inactivated 3T3-J2 cells as described previously.

Donor cells from abdominal skin of a female subject aged 43 years, and maintained in culture according to the above method, were obtained.

### Example 3

### De-Epidermised Dermis (DED) Organotypic Culture

De-epidermised dermis (DED) was prepared as described previously (Rikimaru et al, Experimental dermatology 6, 214-221 (1997)). Briefly, normal adult human skin was cut into 1 cm², heated at 52° C. for 20 min and the epidermis separated from the dermis with forceps. The dermis was depleted of cells by 10 freeze-thaw cycles and irradiated once with 60 Gy. Before fibroblasts were seeded into DEDs, the tissue was placed into 6-well hanging cell culture inserts (Millipore) and equilibrated with DMEM. Different fibroblast preparations (freshly-isolated or cultured) comprising different numbers of cells (1×10⁵ cells or 5×10⁵ cells) were injected into each DED using U-100 insulin syringes (BD) from the epidermis surface. The DEDs were then incubated for 24 h completely submerged in DMEM at 37° C. in a humidified atmosphere with 5% CO₂. Medium was changed to FAD medium with an air-liquid interface, and 1×10⁶ keratinocytes were seeded on top of the DED. DEDs were maintained in culture with FAD medium at an air-liquid interface for 3 weeks with media changes every 48 h. Samples were embedded in OCT prior to sectioning.

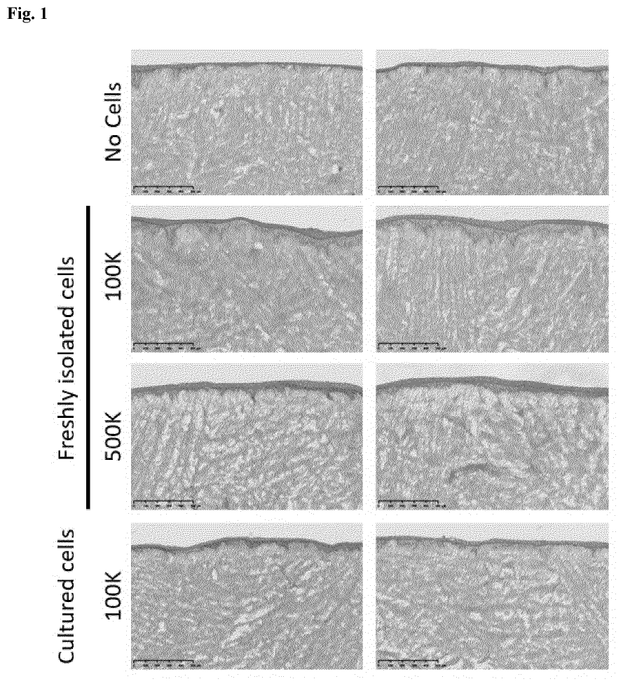

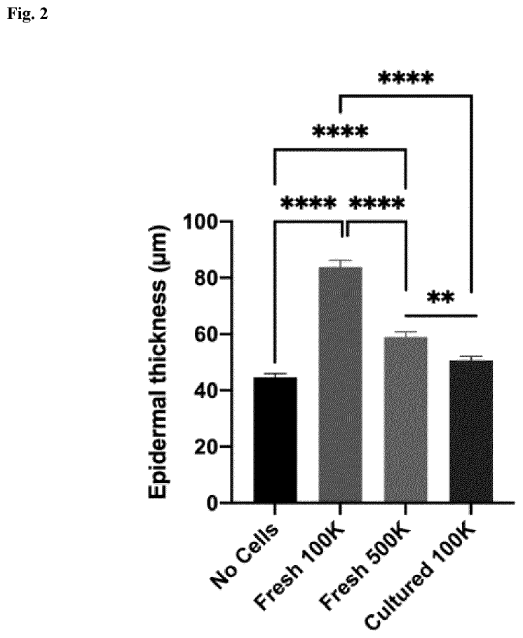

Donor cells obtained by the methods of Example 1 (freshly-isolated fibroblasts) and Example 2 (cultured fibroblasts) were tested in the DED model. The results are shown in FIG. 1A and FIG. 1B. The results show that, surprisingly, freshly-isolated fibroblasts resulted in better epidermal reconstitution than cultured fibroblasts. Even more surprisingly, a lower number of fibroblasts (100,000 freshly-isolated cells) resulted in a greater epidermal thickness than a higher number of fibroblasts (500,000 freshly-isolated cells). This demonstrates that low numbers of freshly-isolated fibroblasts can be used to treat skin conditions characterised by dermal atrophy, without the need for a cell culture expansion step.

### Example 4

### Quantitative RT-PCR Analysis of Gene Expression

Gene expression in freshly isolated dermal fibroblasts (Example 1) was analysed and compared to cultured dermal fibroblasts (Example 2). Total RNA was isolated using the Qiagen RNeasy mini kit (Qiagen) and cDNA was synthesised using the QuantiTect Reverse Transcription kit (Qiagen) or Superscript III First-Strand Synthesis (Thermofisher). Additional RNAse H treatment was completed at the end of the reaction, by adding 1 μl of RNAse H per 30 μl reaction, for 20 min in PCR block at 37° C.

RT-qPCR reactions were performed on CFX384 Real-Time System (Bio-Rad) using the standard protocols for SYBR-Green Master Mix (Life Technologies) using qPCR primers (published or designed with Primer3). Values were normalised to RPL13A and TBP expression levels using the Delta CT method. Each reaction was completed with at least biological triplicates unless otherwise stated.

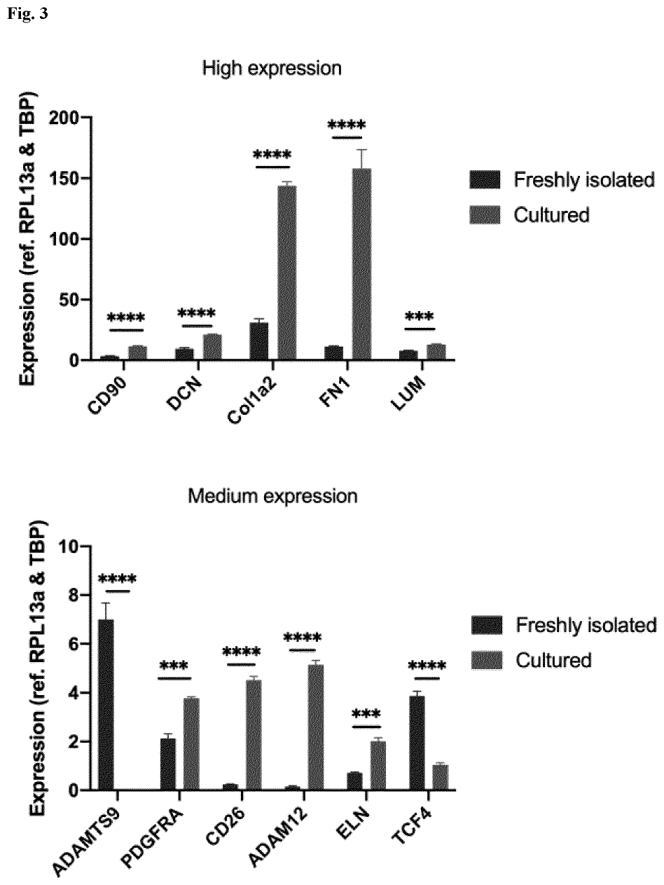

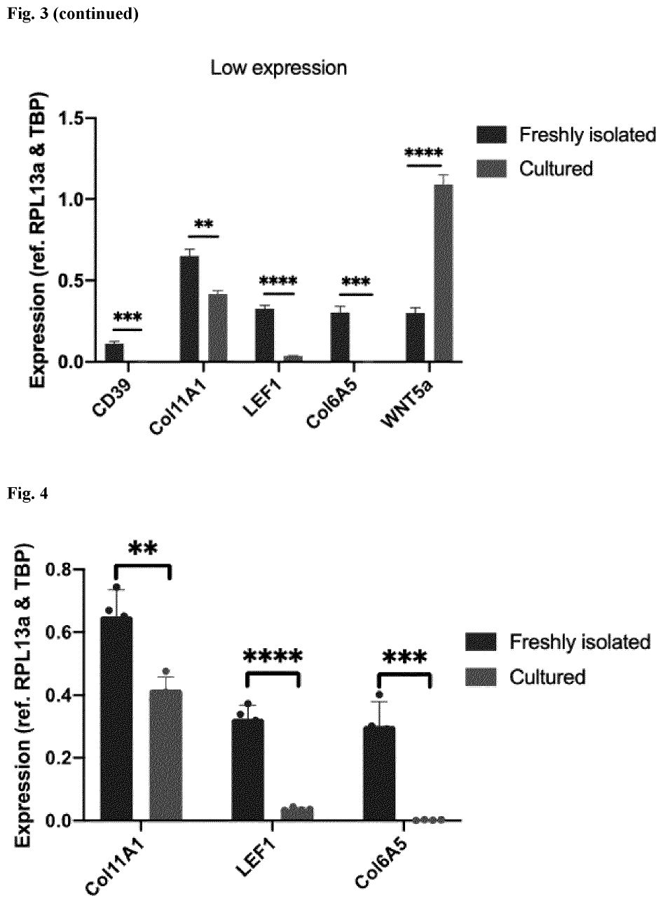

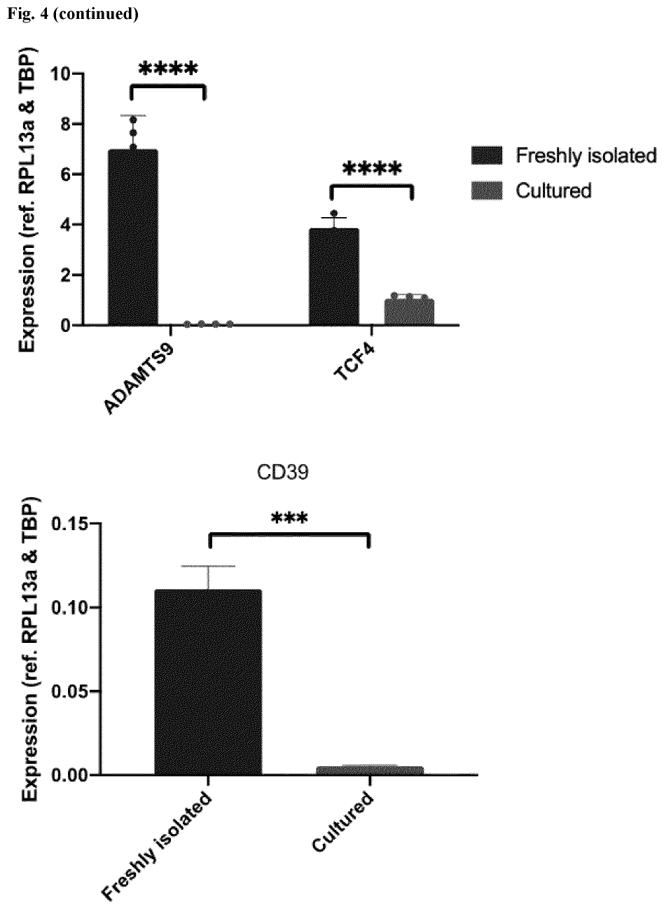

The results are shown in FIG. 3 and FIG. 4. The results shown that following genes are upregulated in freshly-isolated cells compared to cultured cells: ADAMTS9, TCF4, CD39, Col11A1, LEF1 and Col6A5. The following genes are downregulated in freshly-isolated cells compared to cultured cells: CD90, DCN, Col1a2, FN1, LUM, PDGFRA, CD26, ADAM12, ELN and WNT5a.

### Example 5

### Treatment of Atrophic Scarring in the Subject

The cell preparation obtained in Example 1 is used to treat an area of atrophic scarring in the same subject. Areas of administration are sterilised using iodine/chlorhexidine solution. The cell therapy is injected intradermally in doses of 100 μl (containing 100 000 cells) per cm² of atrophic scar tissue.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

### Example 5

### Comparing the Efficacy of Digestion of Human Dermis Using Different Enzymes

The methods of example 1 were repeated using different enzyme combinations to determine the optimal mixture for digestion of the dermis.

A non-sun exposed area of human skin with minimal presence of skin appendages was sterilised with iodine/chlorhexidine and injected with local anaesthetic prior to excision. Skin samples were frozen until use.

After thawing, 6 mm biopsies were taken. After removal of the epidermis, whole, intact dermis samples were incubated in 1 mL digestion medium (100 U/ml penicillin; 100 mg/ml streptomycin in DMEM (high glucose), filter sterilised) supplemented with either: a) Collagenase (ColB, CLSAFB (AFA grade) 300 U/mL (Worthington)), b) Collagenase and DNase I ((Research grade) 1 mg/mL (Roche)) (ColB+DNase I), c) Collagenase, clostrapain and neutral protease (NB4, Nordmark), or d) Collagenase, clostrapain, neutral protease and DNase I (NB4+DNase I). A negative control of PBS alone is shown. Samples were incubated in their respective digestion mixture for 2 hours at 37° C., in a shaking water bath. After two hours the digestion mixture was removed and DMEM supplemented with enzyme-inhibitor solution (500 μl, 0.04 M EDTA in phosphate-buffered saline (PBS)) was added.

At the end of the digestion period, the digested dermis samples were fixed in formal saline and embedded in paraffin and histological sections prepared. Sections of paraffin-embedded digested dermis were treated with xylene to remove wax and rehydrated using graded solutions of ethanol in water. Sections were stained for 2 minutes with Herovici's solution consisting of 50 mL Van Gieson's solution (Sigma), 50 mL 0.05% Methylene Blue solution (Sigma), 10 mL glycerol and 0.5 mL lithium carbonate solution (sat. aq.). Slides were washed for 2 minutes with 1% acetic acid and then dehydrated using graded solutions of ethanol followed by xylene.

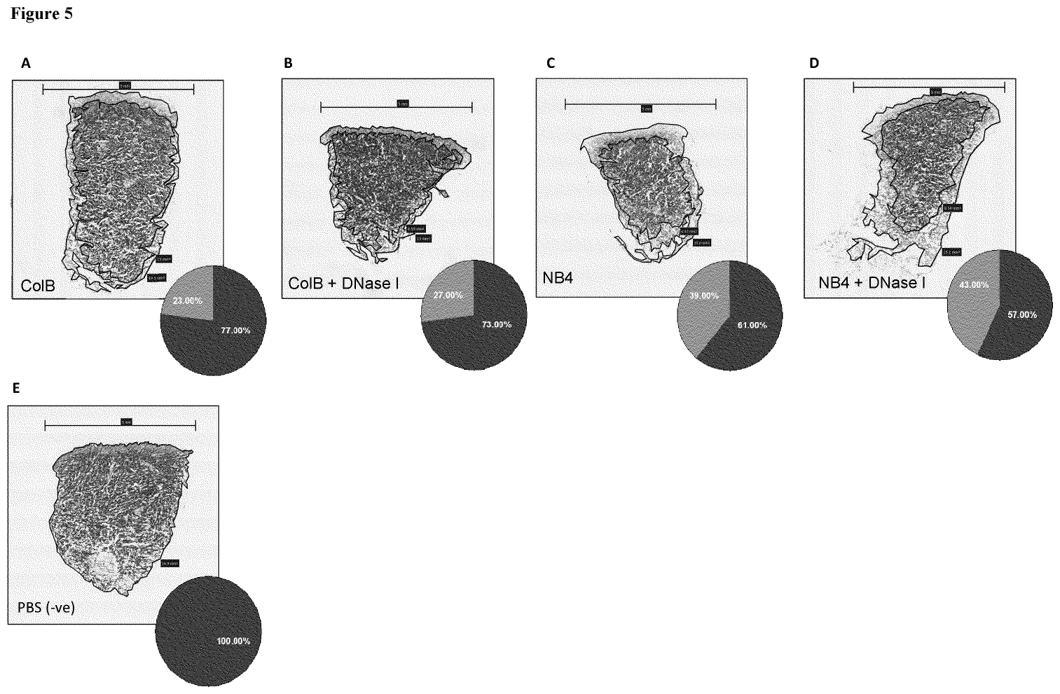

The results are shown in FIG. 5. The purple stained areas contain undigested type I collagen, while the blue areas contain digested collagen. The proportions of purple and blue staining in each section were determined using ImageJ software and are shown in the pie charts.

As demonstrated in FIG. 5, samples treated with a single enzyme (collagenase B) showed 23% digestion of the dermis (FIG. 5A). However, FIG. 5 shows that the level of digestion increased with an increasing number of enzymes. Indeed, FIG. 5B shows that 27% digestion of the dermis was achieved with two enzymes (Collagenase B+DNase I), which indicates a slight increase compared to Collagenase alone. More surprisingly, the level of digestion increased to 39% when using three enzymes (NB4: Collagenase, clostrapain and neutral protease, FIG. 5C), and this was further increased to 43% digestion when using four enzymes (NB4: Collagenase, clostrapain and neutral protease plus DNase I, FIG. 5D).

It therefore appears that more efficient digestion of dermal tissue samples can be achieved by a combination of different enzymes.

We therefore tested whether the differences in digestion efficiency correlated with the number of cells obtained, and whether the cells obtained using multiple enzymes would be viable (i.e., not killed during the digestion process).

To do this, fresh 6 mm human face dermal tissue was prepared by removing the epidermis, macerating the tissue, and incubating the macerated tissue in digestion medium plus either (i) collagenase I (2 mg/mL) and papain (0.15%), or (ii) collagenase I (2 mg/mL), papain (0.15%) and trypsin (0.25%) for 3 hours at 37° C. Enzyme-inhibitor solution (500 μM EDTA in phosphate-buffered saline (PBS)) was added to the resulting cell suspension, filtered through a 100 μm cell strainer, and centrifuged at 300×g, for 5 minutes at 4° C.

The supernatant was discarded and the pellet was washed once with 1.5 ml PBS at at 300×g, for 5 minutes at 4° C. The pellet was resuspended in 0.2 ml PBS/saline and live/dead cells counted using trypan blue.

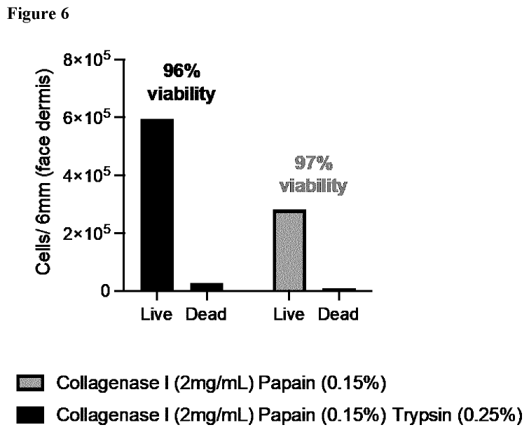

The yield of dermal fibroblasts and their viability is shown in FIG. 6. As can be seen in FIG. 6, the number of cells obtained from the samples was substantially increased when the number of enzymes used for digestion increased from two enzymes, to three enzymes. This is in line with the data obtained from the frozen skin tissue in FIG. 5. Interestingly, the increased number of enzymes did not affect the viability of the cells obtained, with 97% of the cells being viable for the condition where two enzymes were used, and 96% of the cells being viable for the condition where three enzymes were used.

Therefore, we show that combinations of enzymes may be used to increase the number of cells obtained from dermal tissue without any impact on the viability of the cells. Moreover, the combinations of enzymes used in the present invention surprisingly releases a higher proportion of fibroblasts from the tissue, compared to other cell types.

In further aspects, the present invention provides additional embodiments as described in the following numbered paragraphs:
1. A method of preventing or treating a skin condition in a subject, comprising isolating cells comprising primary dermal fibroblasts from a skin sample obtained from the subject, and administering the freshly-isolated cells to skin of the subject.
2. A method according to paragraph 1, wherein the cells are administered to the subject without an ex vivo expansion step.
3. A method according to paragraph 1 or paragraph 2, wherein the cells are administered to skin of the subject within 48 hours, 24 hours or 12 hours of obtaining the skin sample from the subject and/or isolating the cells from the skin sample.
4. A method according to paragraph 1, wherein fewer than 500,000, 250,000 or 150,000 cells are administered per cm² of skin of the subject to be treated.
5. A method according to any preceding paragraph, wherein the cells are administered to the subject in a medium further comprising hyaluronic acid and/or collagen.
6. A method according to any preceding paragraph, wherein the method is a cosmetic method, and/or the skin condition is skin ageing, wrinkling, scarring or atrophy.
7. A method according to any preceding paragraph, wherein the skin sample is obtained from the posterior auricular area, upper arm, thigh or back of the subject, and/or the cells are administered to skin on the face, neck or hands of the subject.
8. A method according to any preceding paragraph, wherein the cells are isolated from the skin sample by a method comprising one or more of the following steps:
   (a) removal of the epidermis from the skin sample;
   (b) enzymatic digestion of dermal tissue; and/or
   (c) separation of cells by cell filtration and/or centrifugation.
9. A method according to paragraph 8, wherein the skin sample is incubated with one or more enzymes selected from dispase, collagenase, DNAse and/or hyaluronidase.
10. A method according to any preceding paragraph, wherein the freshly isolated cells administered to the subject consist of or consist essentially of total dermal cells.
11. A method according to any of paragraphs 1 to 9, wherein the cell isolation step further comprises separating dermal fibroblasts from the skin sample based on expression of one or more cell-surface markers selected from CD31, CD45, CD90 and CD324, preferably CD90.
12. A method according to any of paragraphs 1 to 9 or 11, wherein the freshly isolated cells administered to the subject consist of or consist essentially of unfractionated dermal fibroblasts, preferably having a cell surface phenotype CD45− CD31− CD324− CD90+.
13. A method according to any of paragraphs 1 to 9 or 11, wherein the cell isolation step further comprises separating a subpopulation of dermal fibroblasts from the skin sample based on expression of one or more cell-surface markers selected from CD26, CD39 and CD36.
14. A method according to any of paragraphs 1 to 9, 11 or 13, wherein the freshly-isolated cells comprise or consist of (i) papillary dermal fibroblasts, preferably having a cell surface phenotype CD90+ CD39+ CD26−; and/or (ii) reticular dermal fibroblasts, preferably having a cell surface phenotype CD90+ CD39− or CD90+ CD39+ CD26+.
15. A method according to any preceding paragraph, wherein the cell isolation step further comprises separating dermal fibroblasts or a subpopulation thereof from the skin sample using one or more ligands specific to a cell-surface marker of dermal fibroblasts immobilised on a solid phase, preferably one or more antibodies bound to a magnetic bead or column.
16. A method according to any preceding paragraph, wherein (i) the freshly-isolated cells show increased expression of one or more of the following genes compared to cultured cells: ADAMTS9, TCF4, CD39, Col11A1, LEF1, Col6A5; and/or (ii) the freshly-isolated cells show reduced expression of one or more of the following genes compared to cultured cells: CD90, DCN, Col1a2, FN1, LUM, PDGFRA, CD26, ADAM12, ELN, WNT5a.
17. A method according to any preceding paragraph, wherein at least 100,000, 250,000 or 500,000 primary dermal fibroblasts are isolated per cm² of the skin sample.
18. A method according to any preceding paragraph, wherein the subject is human.
19. A freshly-isolated cell preparation comprising primary dermal fibroblasts obtained from a subject, for use in autologous prevention or treatment of a skin condition in the subject.
20. A freshly-isolated cell preparation for use according to paragraph 19, wherein the preparation is used to treat the subject without an ex vivo expansion step.
21. A freshly-isolated cell preparation for use according to paragraph 19 or paragraph 20, wherein the preparation is used to treat skin of the subject within 48 hours, 24 hours or 12 hours of isolation of cells from the subject.
22. A freshly-isolated cell preparation for use according to any of paragraphs 19 to 21, wherein the preparation comprises fewer than 500,000, 250,000 or 150,000 cells and/or wherein fewer than 500,000, 250,000 or 150,000 cells are administered to the subject.
23. A freshly-isolated cell preparation for use according to any of paragraphs 19 to 22, further comprising hyaluronic acid and/or collagen.
24. A freshly-isolated cell preparation for use according to any of paragraphs 19 to 23, wherein the preparation is used to treat a skin disorder or to promote wound healing.
25. A freshly-isolated cell preparation for use according to any of paragraphs 19 to 23, wherein the preparation is for use by injection to the skin of the subject.
26. A method for isolating cells comprising primary dermal fibroblasts from a skin sample obtained from a subject, comprising:
   (a) removing the epidermis from the skin sample;
   (b) digesting dermal tissue in the skin sample with one or more enzymes; and
   (c) separating cells from the digested sample by filtration and/or centrifugation;
wherein at least 100,000, 250,000 or 500,000 primary dermal fibroblasts are isolated per cm² of the skin sample.
27. A method according to paragraph 26, wherein in step (b) the skin sample is incubated with collagenase type I, DNAse I and/or hyaluronidase IV-S.
28. A method according to paragraph 26 or paragraph 27, further comprises separating dermal fibroblasts or a subpopulation thereof from the isolated cells based on expression of one or more cell-surface markers selected from CD26, CD31, CD39, CD36, CD45, CD90 and CD324, preferably CD90.
29. An isolated cell preparation obtained or obtainable by the method of any of paragraphs 26 to 28.
30. A pharmaceutical unit dosage form comprising freshly-isolated primary dermal fibroblasts in a liquid medium comprising one or more excipients, buffers, or diluents, wherein the unit dosage form comprises fewer than 500,000, 250,000 or 150,000 cells.
31. A pharmaceutical unit dosage form according to paragraph 30, wherein the unit dosage form comprises a sterile solution or suspension suitable for intradermal injection.

1. A freshly-isolated cell preparation comprising primary dermal fibroblasts for use in a method of preventing or treating a skin condition in a subject, wherein the cell preparation is obtained by isolating cells comprising primary dermal fibroblasts from a skin sample obtained from the subject, and the method comprises administering the freshly-isolated cells to skin of the subject.

2. A freshly-isolated cell preparation for use according to claim 1, wherein the cells are administered to the subject without an ex vivo expansion step.

3. A freshly-isolated cell preparation for use according to claim 1 or claim 2, wherein the cells are administered to skin of the subject within 48 hours, 24 hours or 12 hours of obtaining the skin sample from the subject and/or isolating the cells from the skin sample.

4. A freshly-isolated cell preparation for use according to claim 1, wherein fewer than 500,000, 250,000 or 150,000 cells are administered per cm² of skin of the subject to be treated.

5. A freshly-isolated cell preparation for use according to any preceding claim, wherein the cells are administered to the subject in a medium further comprising calcium hydroxyapatite, synthetic poly-lactic acid microspheres, poly (methyl) methacrylate microspheres, polyalkylimide, fibrin, hyaluronic acid and/or collagen.

6. A freshly-isolated cell preparation for use according to any of claims 1 to 5, wherein the cells are administered to the subject in a medium comprising fibrin.

7. A freshly-isolated cell preparation for use according to any preceding claim, wherein the method is a cosmetic method, and/or the skin condition is skin ageing, wrinkling, scarring or atrophy.

8. A freshly-isolated cell preparation for use according to any preceding claim, wherein the skin sample is obtained from the posterior auricular area, upper arm, thigh or back of the subject, and/or the cells are administered to skin on the face, neck or hands of the subject.

9. A freshly-isolated cell preparation for use according to any preceding claim wherein the skin sample has an area of about 0.03 to 3 cm², preferably about 0.1 to 2 cm², more preferably about 0.5 to 1.5 cm².

10. A freshly-isolated cell preparation for use according to any preceding claim, wherein the cells are isolated from the skin sample by a method comprising one or more of the following steps:
(a) removal of the epidermis from the skin sample;
(b) enzymatic digestion of dermal tissue; and/or
(c) separation of cells by cell filtration and/or centrifugation.

11. A freshly-isolated cell preparation for use according to claim 10, wherein the skin sample is incubated with two or more enzymes, preferably two or more enzymes selected from dispase, collagenase, papain, trypsin, thermolysin, pronase, pancreatin, elastase, DNAse and/or hyaluronidase.

12. A freshly-isolated cell preparation for use according to claim 11, wherein one of the two or more enzymes comprises or consists of a collagenase.

13. A freshly-isolated cell preparation for use according to any preceding claim, wherein the percentage of fibroblasts in the freshly isolated cells comprises more than 50%, 60%, 70%, %, 90% or 95% of the total number of cells.

14. A freshly-isolated cell preparation for use according to any preceding claim, wherein the freshly isolated cells administered to the subject consist of or consist essentially of primary dermal fibroblasts.

15. A freshly-isolated cell preparation for use according to any preceding claim, wherein the cell isolation step further comprises separating dermal fibroblasts from the skin sample based on expression of one or more cell-surface markers selected from CD31, CD45, CD90 and CD324, preferably CD90.

16. A freshly-isolated cell preparation for use according to any preceding claim, wherein the freshly isolated cells administered to the subject consist of or consist essentially of unfractionated dermal fibroblasts, preferably having a cell surface phenotype CD45− CD31− CD324− CD90+.

17. A freshly-isolated cell preparation for use according to any preceding claim, wherein the cell isolation step further comprises separating a subpopulation of dermal fibroblasts from the skin sample based on expression of one or more cell-surface markers selected from CD26, CD39 and CD36.

18. A freshly-isolated cell preparation for use according to any preceding claim, wherein the freshly-isolated cells comprise or consist of (i) papillary dermal fibroblasts, preferably having a cell surface phenotype CD90+ CD39+ CD26−; and/or (ii) reticular dermal fibroblasts, preferably having a cell surface phenotype CD90+ CD39− or CD90+ CD39+ CD26+.

19. A freshly-isolated cell preparation for use according to any preceding claim, wherein the cell isolation step further comprises separating dermal fibroblasts or a subpopulation thereof from the skin sample using one or more ligands specific to a cell-surface marker of dermal fibroblasts immobilised on a solid phase, preferably one or more antibodies bound to a magnetic bead or column.

20. A freshly-isolated cell preparation for use according to any preceding claim, wherein (i) the freshly-isolated cells show increased expression of one or more of the following genes compared to cultured cells: ADAMTS9, TCF4, CD39, Col11A1, LEF1, Col6A5; and/or (ii) the freshly-isolated cells show reduced expression of one or more of the following genes compared to cultured cells: CD90, DCN, Col1a2, FN1, LUM, PDGFRA, CD26, ADAM12, ELN, WNT5a.

21. A freshly-isolated cell preparation for use according to any preceding claim, wherein at least 100,000, 250,000 or 500,000 primary dermal fibroblasts are isolated per cm² of the skin sample.

22. A freshly-isolated cell preparation for use according to any preceding claim, wherein the subject is human.

23. A freshly-isolated cell preparation comprising primary dermal fibroblasts obtained from a subject, for use in autologous prevention or treatment of a skin condition in the subject.

24. A freshly-isolated cell preparation for use according to claim 23, wherein the preparation is used to treat the subject without an ex vivo expansion step.

25. A freshly-isolated cell preparation for use according to claim 23 or claim 24, wherein the preparation is used to treat skin of the subject within 48 hours, 24 hours or 12 hours of isolation of cells from the subject.

26. A freshly-isolated cell preparation for use according to any of claims 23 to 25, wherein the preparation comprises fewer than 500,000, 250,000 or 150,000 cells and/or wherein fewer than 500,000, 250,000 or 150,000 cells are administered to the subject.

27. A freshly-isolated cell preparation for use according to any of claims 23 to 26, further comprising one or more dermal fillers.

28. A freshly-isolated cell preparation for use according to claim 27, wherein the one or more dermal fillers comprise collagen, hyaluronic acid, calcium hydroxyapatite, synthetic poly-lactic acid microspheres, poly (methyl) methacrylate microspheres, polyalkylimide, and/or fibrin.

29. A freshly-isolated cell preparation for use according to claim 28, wherein the one or more dermal fillers comprise, consist essentially of or consist of hyaluronic acid, fibrin and/or collagen.

30. A freshly-isolated cell preparation for use according to any preceding claim, wherein the preparation is used to treat a skin disorder or to promote wound healing.

31. A freshly-isolated cell preparation for use according to any preceding claim, wherein the preparation is for use by injection to the skin of the subject, preferably by intradermal injection.

32. A method for isolating cells comprising primary dermal fibroblasts from a skin sample obtained from a subject, comprising:
(a) removing the epidermis from the skin sample;
(b) digesting dermal tissue in the skin sample with one or more enzymes; and
(c) separating cells from the digested sample by filtration and/or centrifugation;
wherein at least 100,000, 250,000 or 500,000 primary dermal fibroblasts are isolated per cm² of the skin sample.

33. A method according to claim 32, wherein in step (b) the skin sample is incubated with two or more enzymes, preferably two or more enzymes selected from dispase, collagenase, papain, trypsin, thermolysin, pronase, pancreatin, elastase, DNAse and/or hyaluronidase.

34. A method according to claim 33, wherein at least one of the two or more enzymes comprises a collagenase.

35. A method according to any of claims 32 to 34, further comprising separating dermal fibroblasts or a subpopulation thereof from the isolated cells based on expression of one or more cell-surface markers selected from CD26, CD31, CD39, CD36, CD45, CD90 and CD324, preferably CD90.

36. An isolated cell preparation obtained or obtainable by the method of any of claims 32 to 35.

37. A pharmaceutical unit dosage form comprising freshly-isolated primary dermal fibroblasts in a liquid medium comprising one or more excipients, buffers, or diluents, wherein the unit dosage form comprises fewer than 500,000, 250,000 or 150,000 cells.

38. A pharmaceutical unit dosage form according to claim 37, wherein the unit dosage form comprises a sterile solution or suspension suitable for intradermal injection.


SEQUENCE LISTING (32 sequences)

SEQ ID NO: 1 - DNA, 20 bp, Artificial Sequence
  forward primer
atcacacccgtcacacatcc

SEQ ID NO: 2 - DNA, 20 bp, Artificial Sequence
  reverse primer
ggggtgatctgtccaacacc

SEQ ID NO: 3 - DNA, 20 bp, Artificial Sequence
  forward primer
cgcgaagacaggcatcaaag

SEQ ID NO: 4 - DNA, 20 bp, Artificial Sequence
  reverse primer
cgtatgtgaaggccgtctcg

SEQ ID NO: 5 - DNA, 19 bp, Artificial Sequence
  forward primer
tgcgctgaaccttcgactg

SEQ ID NO: 6 - DNA, 22 bp, Artificial Sequence
  reverse primer
agcgtggaaattgtctgttctg

SEQ ID NO: 7 - DNA, 20 bp, Artificial Sequence
  forward primer
agccattcgagagtgcaaca

SEQ ID NO: 8 - DNA, 20 bp, Artificial Sequence
  reverse primer
acattgggaagcagaccgtt

SEQ ID NO: 9 - DNA, 20 bp, Artificial Sequence
  forward primer
tcctccaaaccagcaaccag

SEQ ID NO: 10 - DNA, 20 bp, Artificial Sequence
  reverse primer
gcccaacattcctgcatagc

SEQ ID NO: 11 - DNA, 20 bp, Artificial Sequence
  forward primer
gctgattcctgggagcacat

SEQ ID NO: 12 - DNA, 20 bp, Artificial Sequence
  reverse primer
atgtagcccaaagtccagcc

SEQ ID NO: 13 - DNA, 23 bp, Artificial Sequence
  forward primer
tggtgatcagaatcagaagttcg

SEQ ID NO: 14 - DNA, 23 bp, Artificial Sequence
  reverse primer
aggagagttgagaattgggaatc

SEQ ID NO: 15 - DNA, 20 bp, Artificial Sequence
  forward primer
tcccgaaccaacttcaccag

SEQ ID NO: 16 - DNA, 20 bp, Artificial Sequence
  reverse primer
gatgccctcacacttgacca

SEQ ID NO: 17 - DNA, 20 bp, Artificial Sequence
  forward primer
agaagttcctgatgaccgcg

SEQ ID NO: 18 - DNA, 20 bp, Artificial Sequence
  reverse primer
cgaagatggcattgacagcg

SEQ ID NO: 19 - DNA, 20 bp, Artificial Sequence
  forward primer
aggacaagaaacacgtctgg

SEQ ID NO: 20 - DNA, 22 bp, Artificial Sequence
  reverse primer
ggtgatgttctgagaggcatag

SEQ ID NO: 21 - DNA, 20 bp, Artificial Sequence
  forward primer
ttacggtggcaactcaaatg

SEQ ID NO: 22 - DNA, 21 bp, Artificial Sequence
  reverse primer
cagtgtggtctgtgcagaaag

SEQ ID NO: 23 - DNA, 20 bp, Artificial Sequence
  forward primer
gtggtaccagtggccagtac

SEQ ID NO: 24 - DNA, 20 bp, Artificial Sequence
  reverse primer
ttgggtagctttcagggcag

SEQ ID NO: 25 - DNA, 20 bp, Artificial Sequence
  forward primer
ttctgaactcacggtggctg

SEQ ID NO: 26 - DNA, 20 bp, Artificial Sequence
  reverse primer
gctgcatcgggtccacataa

SEQ ID NO: 27 - DNA, 19 bp, Artificial Sequence
  forward primer
agtggcacggcaacacatt

SEQ ID NO: 28 - DNA, 19 bp, Artificial Sequence
  reverse primer
agagcttctatcccgatga

SEQ ID NO: 29 - DNA, 20 bp, Artificial Sequence
  forward primer
gtaggcgtggaagtgtggaa

SEQ ID NO: 30 - DNA, 20 bp, Artificial Sequence
  reverse primer
atgctcatgattggggccat

SEQ ID NO: 31 - DNA, 20 bp, Artificial Sequence
  forward primer
tcctggaattggaggcatcg

SEQ ID NO: 32 - DNA, 19 bp, Artificial Sequence
  reverse primer
aactactcccgggccaaag